(19) United States
(12) Patent Application Publication
(10) Pub. No.: US 2018/0169009 A1
(43) Pub. Date: Jun. 21, 2018

(54) NANOBIOCOMPOSITE COMPOSITIONS AND METHODS

(71) Applicants: Patrick JOHNSON, Albuquerque, NM (US); C. Jeffrey BRINKER, Albuquerque, NM (US); Eric CARNES, Albuquerque, NM (US); Graham TIMMINS, Albuquerque, NM (US); Pavan MUTTIL, Albuquerque, NM (US); STC.UNM, Albuquerque, NM (US)

(72) Inventors: Patrick Johnson, Albuquerque, NM (US); C. Jeffrey Brinker, Albuquerque, NM (US); Eric Carnes, Albuquerque, NM (US); Graham Timmins, Albuquerque, NM (US); Pavan Muttil, Albuquerque, NM (US)

(21) Appl. No.: 15/577,572

(22) PCT Filed: Jun. 1, 2016
(86) PCT No.: PCT/US2016/035289

§ 371 Date: Nov. 28, 2017

Related U.S. Application Data
(60) Provisional application No. 62/168,991, filed on Jun. 1, 2015.

(51) Int. Cl. A61K 9/00; A61K 9/16; A61K 47/54; C08L 23/22; A61K 9/12; A61K 9/127

(52) U.S. Cl. CPC A61K 9/0073; A61K 9/1617; A61K 9/1271; C08L 23/22; A61K 9/12; A61K 47/543

(57) ABSTRACT

This disclosure describes compositions, vaccine, and methods that involve a biocomposite material Generally, the biocomposite material includes a cell and a lipid-silica matrix at least partially encapsulating the cell. In some cases, the cell can be viable but not culturable (VBNC). In some cases, the lipid-silica matrix includes a dried sol and/or possesses ordered nanostructure.

### CROSS-REFERENCE TO RELATED APPLICATION

This application claims priority to U.S. Provisional Patent Application No. 62/168,991, filed Jun. 1, 2016, which is incorporated herein by reference.

### GOVERNMENT FUNDING

This invention was made with government support under AI081015 awarded by the National Institutes of Health, AI081090 awarded by the National Institutes of Health, and FA9550-01-1-0066 awarded by the Air Force Office of Scientific Research. The government has certain rights in the invention.

### SUMMARY

This disclosure describes, in one aspect, a biocomposite material. Generally, the biocomposite material includes a cell and a lipid-silica matrix at least partially encapsulating the cell.

In some embodiments, the cell is viable but not culturable (VBNC).

In some embodiments, the cell comprises a pathogen.

In some embodiments, the lipid-silica matrix comprises a dried sol.

In some embodiments, the lipid-silica matrix possesses ordered nanostructure.

In another aspect, this disclosure describes a method of making a biocomposite.

Generally, the method includes providing a cell, aerosolizing a mixture of lipid and silica precursor, combining the cell and the aerosolized mixture, thereby allowing the aerosolized mixture to at least partially encapsulate the cell, and allowing at least a portion of the aerosolized mixture to evaporate.

In some embodiments, allowing at least a portion of the aerosolized mixture to evaporate induces evaporation self-assembly of the lipid and silica precursors in the aerosolized mixture.

In some embodiments, the method can further include drying the partially-encapsulated cells to form a powder.

In another aspect, this disclosure describes a method of evaluating the effects of a test compound on a cell. Generally, the method includes providing any embodiment of the biocomposite material summarized above, contacting the biocomposite material with the test compound, and evaluating at least one effect of the test compound on the cell.

In some embodiments, the cell can be a pathogen.

In another aspect, this disclosure describes a vaccine that includes any embodiment of the biocomposite material summarized above.

In some embodiments, the biocomposite material may be aerosolized.

In yet another aspect, this disclosure describes a method of treating a subject having or at risk of having a condition treatable with a biocomposite material. Generally, the method includes administering any embodiment of the vaccine summarized immediately above to a subject having or at risk of having a condition treatable by the biocomposite material.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Schematic of the spray drying process using a Büchi B-290 mini spray drier for the production of lipid-silica nanobiocomposites. Solutions of cells in liquid suspension and lipid-silica precursors are mixed in scintillation vials (A) and dispensed into the sprayer nozzle via peristaltic pumps with mixing immediately prior to injection with a Y valve (B). This mixture is aerosolized by the heated nozzle in a sheath of N₂ gas (C). Droplets are approximately 10 μm to 100 μm in diameter and include cells, lipid (an inhomogeneous mixture of free, micellular, or liposomal lipids), silica precursors, and solvent (D). The droplet size can be varied by changing the ratio of the N₂ gas flow rate to that of the liquid feed rate. (E) Lipids organize silica precursors into an ordered nanostructure as the solvent evaporates during Evaporation Induced Self Assembly (EISA). (G) Particles are fully dried before entering the cyclone (F) and flow through cyclone vortex into the collection chamber. An aspiration vacuum pump (Vac.), pulls a vacuum on the assembly, exhausting N₂.

FIG. 2. Particle morphology can be tuned depending on the spray drying parameters listed in Table 1 to yield large or small particles with varying percentages of hollow particles. (A-C) Large particles and/or aggregates prepared with Process A can contain multiple red-fluorescent cells (A, inset) and particles can range from approximately 10 μm to 30 μm or greater (B, C). (D-F) Individual particles prepared with higher temperatures in Process B demonstrate smaller particles, which are more disperse, less prone to aggregation, and contain a subpopulation of hollow particles. (G-I) Lower feed rates reveal a higher fraction of hollow particles with a mixed size distribution. An optical slice of <0.5 μm allows for the cross-sectional visualization of hollow particles (G). Samples were prepared using a green fluorescent lipid, which extends throughout the particle. The scale bar is 5 m.

FIG. 3. Fluorescence confocal images of biocomposites demonstrate discrete, spray-dried particles that fully encapsulate cells or cell surrogates. (A) 1 μm green fluorescent latex beads were used to provide a baseline for spray-dried particles. Encapsulated red fluorescent bacteria (K12 E. coli constitutively expressing DsRed-Express 2) are observed in collapsed merged Z-stack images of a typical large (B) and small (C) particle. Particles are shown to fully encapsulate cells as confirmed with three-dimensional z-stack sectioning of the same large (D) and small (E) particle, for which the optical slices are 0.4 μm and 0.5 μm, respectively, and the total z-scan depths were 13.9 μm and 6.0 μm, respectively.

FIG. 4. Transmission electron microscopy analysis of biocomposites reveals that particles possess ubiquitous nanostructure that is independent of the spray drying parameters and extends throughout solid and hollow regions. (A) A typical particle from a sample prepared according to Process A with 60° C. inlet temperature and 3.5 mL/min feed rate has a well-defined nanostructure that extends throughout the bulk of the particle (bottom). (B) A group of particles prepared according to Process D with 90° C. inlet temperature and 2.5 mL/min feed rate include a cluster of solid (stars) and hollow particles (arrows). The previously observed nanostructure is found within the perimeter of the hollow shells (bottom, red) and extends into solid regions (bottom, green). (C) Biocomposites were prepared with latex beads, which appear dark grey and are fully encapsulated by surrounding particle. Zoomed images show nanostructure throughout the bulk of the particle (bottom, green) and interfacing directly with the bead surface (bottom, red). (D) Spray-dried particles containing E. coli were prepared for TEM following the same technique as with spray-dried beads and are found to have similar bulk nanostructure (bottom, green) that interfaces coherently with the cell (bottom, red). E. coli are stained prior to spray drying with electron dense osmium tetroxide, which binds to the lipids within the bacterial membrane and provides contrast within the electron beam.

FIG. 5. Fluorescence Recovery After Photobleaching of biocomposites prepared with 1% fluorescent lipid (w/w of total lipid fraction) reveals a fluid lipid layer that extends throughout the bulk of the particle that is independent of process parameters and retains fluidity for >18 months. (A) A representative recovery curve showing the recovery profile of a particle prepared under Process A. (B, C) Fluorescence recovery image series of regions that were bleached on a large particle from Process A (B) and many small particles from Process D (C). The green channel exhibits a noticeable recovery whereas the red channel remains quenched. The particles containing cells exhibit high initial fluorescence in both green (fluorescent lipid) and red (RFP) channels and both channels are nearly fully quenched after bleaching with a high intensity laser (yellow dotted circle). The scale bar is 5 μm. (D) The mobile and immobile fractions indicate that 70-85% of the photobleached molecules are in the mobile phase and contribute to the fluorescence recovery. (E) Lipid recovery is observed by tracking the diffusion time, t_{D}, the time to recover of one-half of the final recovered fluorescent intensity.

FIG. 6. Young's modulus and hardness of biocomposites are examined with nanoindentation and found to be 13.0±1.0 GPa and 1.4±0.1 GPa, respectively (n=10), using the standard Oliver-Pharr analysis (Oliver et al., 2004, J. Mater. Res. 19:3-20). The epoxy-embedded sample from which TEM samples were previously microtomed was used to perform indentation studies as described in FIG. 16. Nanoindentation was performed on this sample using a nanoindentor (TRIBOINDENTER, Hysitron, Inc., Eden Prairie, Minn.) with a three-sided pyramidal Berkovich tip with 50-nm radius. Pictured are topography images (A-C) achieved by scanning the nanoindentor tip (top) and ‘gradient reverse’ images based on the derivative of the topography image (bottom) of one particle prepared by Process A before indentation (wide field scan, left; zoom, center) and after 2× indentations (right). The inset shows a typical area before and after indentation. Indentations were taken on multiple particles from different regions of the sample. (D) The Young's Modulus and Hardness of silica biocomposites compared to surrounding epoxy (n=10 and 29 respectively).

FIG. 7. An ATP-based viability assay of aged biocomposites indicates that encapsulated cells are viable for at least eight months with less than 1-log₁₀ loss in ATP for samples stored at 40° C. and 0% Relative Humidity (RH) and that this long-term viability is independent of process parameters. Spray-dried samples were prepared, split into 5-10 mg aliquots and stored at 25° C./60% RH, 40° C./75% RH, or 40° C./0% RH. Samples were periodically removed from aging and resuspended in water to a 25 μL/mg dilution. The solution was thoroughly mixed and 25 μL was added to a 96-well plate in duplicate. The luciferase reagent was prepared according to product literature, 50 μL reagent was added to each sample well, and the plate was analyzed on a luminometer (Tecan Group Ltd., Mannedorf, Switzerland). The data was normalized to ATP standards and converted to mols ATP. For samples stored at 40° C. and 0% RH, there was <1-log₁₀ loss in viability after eight months aging, a significant improvement over reported values of ˜4-log₁₀ loss of storage under the same conditions for only 30 days. This data was compared to samples stored at 4° C., which was found to have no distinguishable change in ATP after eight months (FIG. 17).

FIG. 8. Aged cells can be grown in liquid culture and demonstrate some characteristics of bacterial persistence. Here, the same, known concentration of cells is added to each well of a 96-well plate and the plate is sealed and incubated with shaking for up to two months. If it occurs, the growth in a well takes up to 24 hours to go from null to maximal growth and is observed by monitoring RFP bacterial fluorescence. The majority of growth occurs within the first several days of incubation, but it can continue for up to two weeks, after which point little growth was observed. (A) Growth of cells per well after dry aging for up to 36 weeks prior to incubation. The well-growth frequencies cap out at 96 due to the number of wells per plate. (B) Representative fluorescence images taken with a digital camera with backlighting from a UV-transilluminator (blue) and a plate reader (black and white) of 96-well plates that were used to conduct the experiment highlight wells that exhibit growth (bright). (C) The maximum weeks of aging where 50% regrowth/resuscitation remains possible as a function of the average number of cells/well.

FIG. 9. Evolution of the silica Precursor Solution as it undergoes a condensation aging process during which mixed phases condense into a single, homogeneous phase. After combining the precursors as described in the methods section, the solution was homogenized with shaking and the vial was placed on a rocking platform for up to 1 hr and imaged periodically. (A) The solution immediately after addition of reagents and shaking. (B-D) mixed phases are observed within the bulk of the solution and collect at the bottom of the vial when not subjected to mixing/rocking. (E) The solution condenses fully within about 60 minutes of initial mixing and is colorless, translucent, featureless, and unchanging. Nanostructure and ATP experiments indicate that this solution remains usable for preparation of nanobiocomposites (NBCs) for more than one hour after complete condensation.

FIG. 10. Representative transmission electron microscopy images of lipid/silica mesophase particles formed by so-called ‘aerosol-assisted’ evaporation induced self-assembly. All particles are solid and roughly spherical and exhibit ordered nanostructures consistent with hexagonal mesophases. Sample were prepared using the identical Precursor Sol as for spray-drying and the aerosol-assisted EISA approach as previously described (Lu et al., 1999, Nature 398:223-226). The aerosol reactor consisted of an aerosol generator (TSI, Inc., Shoreview, MN) operated with 207 kPa N₂ as the carrier gas and a seven zone tube furnace with temperatures 150° C., 170° C., 170° C., 270° C., 410° C., 170° C., and 115° C. Powders were collected on a 0.2 μm SUPOR membrane (Pall Corp., Port Washington, N.Y.) at 115° C.

FIG. 11. (A) Particle sizing with Laser Diffractometry (LD) and separation by Mass Mean Aerodynamic Diameter (MMAD) with a cascade impactor indicate that particles with MMAD as low as 0.54 μm contain cells. (A) Particle sizing using LD demonstrates disperse, discrete particles with 50% of the particles falling between approximately 6 μm to 14 μm. LD of spray-dried powders demonstrates particle size distribution with a median particle size (×50) of approximately 12 μm. Shown is a typical particle size curve. Plotted on the dual y-axis are the cumulative volume (q(x)) and the volume frequency (Q(x)) particle size distributions. Spray dried samples were prepared as previously described, 1 mg powder was suspended in 1 mL of acetonitrile, vortexed for 30 seconds to distribute particles, and 100 μL was pipetted into an LD cuvette containing acetonitrile. (B) Particles were separated by their MMAD using a cascade impactor and a sample from each well was imaged with a confocal microscope. Fluorescence (top) and DIC-fluorescence merge (bottom) images indicate that particles with MMADs small as 0.5 μm can contain cells. Particles correspond to wells 1-7 of the impactor and diameters of 11.70 μm, 6.40 μm, 3.99 μm, 2.30 μm, 1.36 μm, 0.83 μm, and 0.54 μm, respectively.

FIG. 12. Confocal image gallery of merged fluorescence and DIC channels for a large biocomposite particle. The 35 images span 13.0 μm for a total optical slice of 0.37 μm per image.

FIG. 13. Confocal image gallery of merged fluorescence and DIC channels for a small biocomposite particle. The 13 images span 6.0 μm for a total optical slice of 0.46 μm per image.

FIG. 14. Yeast can be incorporated into spray dried biocomposites and maintain intact RNA after two weeks of aging. (A) Yeast were stained with a green fluorescent cell-permeable, nucleic acid dye (Syto 9, Invitrogen Corp., Carlsbad, Calif.) verifying the integrity of the cell wall and demonstrating the permeability of the lipid/silica matrix to small molecule dyes with high specificity to cells and low interaction with matrix. (B) An RNA purification assay was developed that allows for the removal of intact RNA from biocomposite powders demonstrating RNA levels of between 33 ng/μL and 132 ng/μL of extract. Spray dried biocomposites containing Saccharomyces cerevisiae were stored for 0 days, 3 days, or 14 days at 25° C./60% RH or 40° C./0% RH and processed with an RNA extraction assay designed uniquely for these lipid/silica biocomposites. Purified RNA samples were analyzed on a bioanalyzer to determine the total RNA concentration. Gel electrophoresis was used to separate the individual bands and compared across the samples. Results indicate that the unaged sample contains well-defined RNA pattern with strong bands corresponding to the 16S small and the 30S large bacterial ribosomal subunits. The ladder concentration was 150 ng/μL.

FIG. 15. (A) X-Ray Diffraction (XRD) plots of samples prepared under varying conditions. The baseline condition is referred to as Process A (Inlet temp: 60° C.; feed rate: 3.5 mL/min; gas rate: 60 L/hr) and the varied conditions are 1) age of the Precursor Sol, 2) increased and decreased rate of the inlet feed solution (2.5 mL/min and 4.5 mL/min, respectively), 3) decreased carrier gas flow rate (30 L/hr), and 4) two degrees of elevated inlet temperature (90° C. and 120° C. respectively). Results demonstrate no noticeable difference between the nanostructures indicating preserved nanostructural features independent of processing parameters (consistent with TEM) and to have a lower angle and broader diffraction peak as compared to thin film samples, indicating a larger characteristic d-spacing and moderately less order. Powders were collected from spraying chamber and analyzed as described in the methods section with an X'PERT Pro diffractometer (PANalytical, Inc., Boulder, Colo.) using CuKα radiation with λ=1.15418 Å. (B) XRD 20 and corresponding d-spacings of various powders and thin or thick cast films formed by EISA procedures in this study. (C) XRD plot of samples from (B). Nanobiocompsite powders were prepared according to Process A or aerosol-assisted EISA (FIG. 10) and thin and thick film samples prepared by EISA via spin-coating or casting. Nanobiocompsite powders were found to have a characteristic peak at 2.88° 2θ, which is lower that of powders prepared by aerosol-assisted EISA and thin or thick cast films prepared by spin-coating or casting. Differences are attributed to the rapid drying time of spray-dried samples (less than one second) as compared to aerosol-EISA powders (1-10 seconds), spin-cast (1-10 seconds), or thick-cast samples (10 seconds to 10 minutes, depending on Precursor Sol volume)). Here, two different aerosol routes were analyzed as previously described (Lu et al., 1999, Nature 398:223-226) to determine the nanostructural properties of sub-micron particles. Thin films were prepared using Precursor Sol, lipid, and bacteria as described above and 100 μL of solution is pipetted onto a substrate (1 cm² Si wafer or a round cover glass) without N₂ by spin-coating at ambient conditions or with N₂ by spin coating within a N₂-rich chamber (Laurell spin coater, model WS-400Bz-6NPP-Lite) at 1,000 RPM for 30 seconds. Thick-cast films were prepared by adding 1 mL of Precursor Sol, lipid, and bacteria as described above onto a 1 cm² Si wafer or a round cover glass and the solvent is allowed to evaporate. (D) Grazing Incidence Small Angle X-ray Scattering (GISAX) data for thin films (top) and thick films (bottom) films demonstrate nearly identical nanostructures.

FIG. 16. Schematic depicting the preparation of samples used for TEM, SEM backscatter and nanoindentation studies. (A) Two samples were prepared from the standard TEM preparation technique: a thin film (60 nm width) used for TEM studies and the remaining bulk sample, which was characterized with SEM and nanoindentation. Following standard TEM sample preparation, a 60-nm thin film is microtomed from the surface of the sample and used in TEM analysis. The remaining sample is used for backscatter SEM imaging (B) and nanoindentation studies (FIG. 6) with no additional sample preparation (i.e., no surface metal coating) and highlights the surface morphology observed in TEM analysis (FIG. 4). This image was used as a guideline for seeking particles to analyze with nanoindentation and can be compared to the surface morphology observed in FIG. 6A. (C) Table elaborating on room temperature and 40° C. storage effects on structural properties of cell-directed assembly films. Thin and thick films were prepared as described earlier and were subjected to a nanoindentation study. The contact depth of the probe was kept between 65 nm and 100 nm, which, for the thin film, was a sizable fraction of the film depth. Thus, the thick-film data provides a more accurate depiction of film structural and hardness properties.

FIG. 17. (A, B) E. coli immediately prior to spray drying show 96% viability as determined using Baclight Live/Dead assay (Molecular Probes Co., Eugene, Oreg.). Cells that were treated with 1% bleach for 10 minutes are 0% viable. (C) Percentage of wells showing viability/resuscitation as a function of incubation time in buffer/FBS for cells encapsulated in nanobiocomposites for two weeks and dispensed at an average concentration of 100 cells per well. (D) Fresh and eight-month aged nanobiocomposites were analyzed using a luminescence ATP assay, which shows unchanging ATP levels after dry aging at room temperature. (C) For the live/dead study, E. coli were grown overnight to 1.75 OD₆₀₀, washed three times in PBS, and resuspended in PBS. One batch was left at room temperature for 30 minutes and the other was mixed with standard household bleach to a total concentration of 1% bleach for 10 minutes followed by 3× wash in PBS. The 30-minute aging condition was used to simulate the maximum amount of time that cells would remain in buffer prior to spray drying. The live/dead staining was performed per manufacturer recommendations. The percentage of live cells was determined by counting 1000+ cells and determining the number of green (live) versus red (dead) cells.

FIG. 18. Analysis of resuscitation frequency (regrowth) in liquid media as a function of bacterial number and of aging times in a biocomposite. (A) 10⁰ cells/well, (B) 10¹ cells/well, (C) 10² cells/well, (D) 10³ cells/well, (E) 10⁴ cells/well, (F) 10⁵ cells/well.

FIG. 19. Schematic of an exemplary nonbiocomposite aerosolization process. (A) Silica precursors are mixed with lipids and bacteria cells in solution to form the feedstock solution, which is dispensed into the apparatus immediately prior to aerosolization. This is continuous feed process achieved using separate supplies of silica/lipid solution and cells in solution via a multi-channel syringe pump. The two solutions are mixed immediately prior to aerosolization. (B) The feedstock solution is forced through an aperture plate consisting of 1,000+ holes which vibrates at 100,000+ Hz resulting in a suspension of ˜10⁸ droplets per second. Evaporation Induced Self Assembly drives the formation of ordered, lipid-templated silica nanostructures, which encapsulate the bacteria. (C) The final product is a homogeneous, dry powder consisting of 2-3 μm particles containing cells.

FIG. 20. Schematic of the aerosolization assembly. (A) Silica and lipid precursors are loaded into Syringe 1 and washed bacterial cells in buffer are loaded into Syringe 2. The syringes are mounted on and controlled from a multi-syringe pump set to dispense 0.15 mL per minute per channel. (B) The solutions are combined via a Y connection immediately prior to dispensing into the nebulizer unit. Solution mixing occurs rapidly, ensuring a homogeneous solution delivered to the nebulizer (C). Adapted from product manual to include a second syringe for dispensing of multiple sensitive reagents.

### DETAILED DESCRIPTION OF ILLUSTRATIVE EMBODIMENTS

This disclosure describes methods and articles involving biocomposites that contain living cells within a three-dimension lipid-silica matrix. In some cases, the lipid-silica matrix can exhibit ordered nanostructure. This disclosure, in one aspect, describes a spray drying process enabling the large-scale production of functional biocomposites. The spray drying process works with multiple cell types and results in dry powders exhibiting a unique combination of properties including, for example, highly ordered 3D nanostructure, extended lipid fluidity, tunable macro-morphologies, tunable aerodynamic diameters, and/or unexpectedly high physical strength.

In another aspect, this disclosure describes a quantitative method of determining resuscitation frequencies of viable but not culturable (VBNC) cells. This method was used to determine that after 36 weeks in a biocomposite-induced VBNC state, less than 1 in 10,000 cells underwent resuscitation. The biocomposite platform production of large quantities of VBNC cells is of interest for research in bacterial persistence and screening of drugs targeting such cells. The biocomposites described herein also may enable long-term preservation of living cells for applications in the packaging and delivery of live-cell vaccines.

This is the first demonstration of spray-dried biocomposite materials in which living cells are encapsulated within a protective lipid-silica nanostructured matrix by evaporation-induced cell directed assembly (CDA). The spray-dried biocomposite materials are shown to be mechanically robust with controlled structures spanning the nanoscale to microscale regimes depending on spray drying conditions. The bio-functionality of biocomposite-encapsulated cells (e.g., E. coli) is preserved for months as shown by ATP and re-growth assays. Based on ease of processing and the ability to engineer both the nano-bio interface and macroscopic aggregate morphology/aerodynamic diameter, the biocomposite spray drying process can have broad applicability in pharmacology, cell-based sensing, microbial fuel cells, vaccines, and/or fundamental studies of biology at the individual and multiple cell scales.

Generally, the biocomposites include a lipid-silica matrix formed from lipid and silica precursors. Suitable silica precursors include, for example, a silicate (e.g., silicon oxide or silicon hydroxide prepared by any form of sol-gel processing from aqueous silica); a silicon alkoxide such as, for example, where hydrolysis and condensation of the silica is conducted at an acidic pH range (e.g., pH 2-3); SiCl₄; or an organosilane such as, for example, one in which the metal framework is doped by another metal at a low concentration. Suitable doping metals include, for example, Group III metals (e.g., B, Al Ga, In), Group IV metals (e.g., Ge, Sn Pb), transition metals, Group I metals, Group II metals, and rare earth metals.

Suitable lipid precursors include zwitterionic lipids such as, for example, di-C6 zwitterionic lipids (e.g., having two C6 tails), diC8 zwitterionic lipids, and diC10 zwitterionic lipids having some degree of water solubility, and mixtures thereof. In some embodiments, the lipid precursor can include single chain lipids.

### Spray Drying of Lipid-Silica Nanobiocomposites

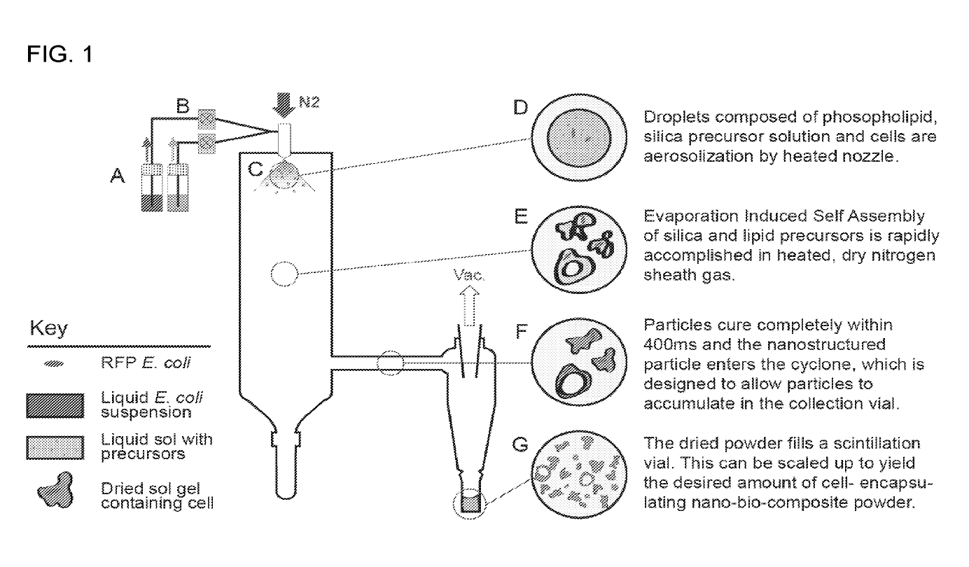

Evaporation Induced Self Assembly (EISA) of highly ordered lipid-silica matrices encapsulating cells is an established technique for the preparation of living biomaterials. FIG. 1 illustrates a modified process adapted to spray drying using a commercial bench-top spray drier fitted with a custom collection receptacle (FIG. 1). The spray dry process involves the delivery of liquid precursors to a heated nozzle. In the exemplary embodiment illustrated in FIG. 1, the heated nozzle injects the solution into a nitrogen gas sheath that is maintained by vacuum aspiration through the top of the cyclone. The solution dries within the heated sheath gas and the dried powder collects into a vial. By systematic modulation of the primary control parameters, the average liquid droplet size can be varied over the approximate range from less than 10 μm to over 100 μm with rather broad particle size distributions and the dried aggregate morphology can be varied from compact irregular particles to hollow, more spherical shapes.

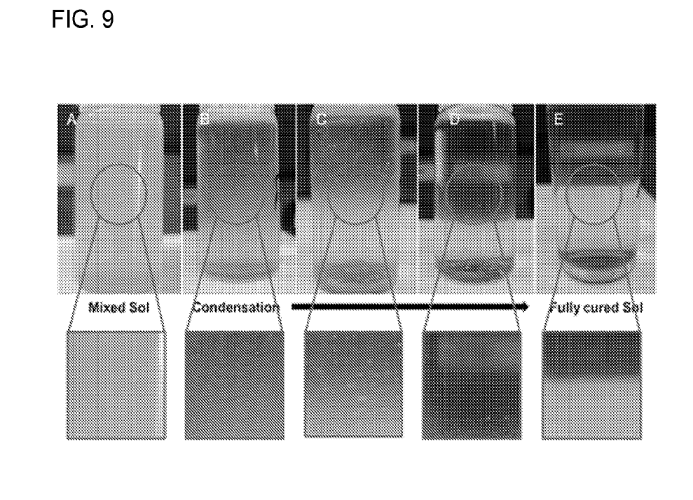

Lipid-silica precursor solutions (termed Precursor Sol) were prepared as previously described (Baca et al., 2006, Science 313:337-341) and are summarized in the EXAMPLES section below and FIG. 9. To assess the encapsulation efficiency and distribution of cells within spray-dried powders, initial studies employed E. coli bacteria expressing Red Fluorescent Protein (RFP) in liquid culture or control green fluorescent latex beads of comparable size and surface charge as E. coli as a control. The initial spray drying parameters—nozzle temperature, solution feed rate, gas flow rate, and vacuum aspiration level—are shown in Table 1.

TABLE 1
Process parameters tested within this study define
the limits of powder formation.

| User-defined | | |
|---|---|---|
| process | | |
| parameters | Range tested | Notes |
| Nozzle temperature | 60° C.-120° C. | <60° C.: no powder formation |
| | | >120° C.: not tested |
| Solution feed rate | 2.5-4.5 mL/min | <2.5 mL/min: not tested |
| | | >4.5 mL/min: no powder formation |
| Sheath gas flow rate | 30-60 L/hr | <30 L/hr: no particle formation |
| | | 60 L/hr: maximum flow rate |
| Vacuum aspiration | 90-100% | <90%: no particle collection |
| level | | 100%: maximum level |

*The outlet temperature for all processes tested remained between 30 and 45° C. depending on the inlet temperature and the solution feed rate.

E. coli cells in phosphate buffered saline (PBS) and the Precursor Sol were dispensed into separate scintillation vials and delivered to the heated nozzle with two peristaltic pumps—one for each solution (FIG. 1A). The solutions were combined immediately prior to injection into the drier nozzle (FIG. 1B). This method minimizes cell contact time with Precursor Sol constituents (˜15% ethanol v/v and pH 3) allowing for increased cell viability. Aerosolization of the solution (FIG. 1C) occurs in a sheath of heated nitrogen gas (60° C.−120° C. as tested), where the aerosolized liquid droplets (FIG. 1D) were estimated to range from 50 μM to 100 μM in diameter, depending on the processing parameters. Solvent evaporation from the droplets within the heated nitrogen stream progressively increases the concentration of non-volatile Precursor Sol constituents and drives self-assembly of the droplets into periodic, ordered lipid-silica mesophases in a manner related to aerosol assisted EISA (FIG. 1E). Ensuing evaporation and thermally driven condensation of the soluble silica precursors solidifies the particles (FIG. 1F) as they enter the cyclone, exit the gas flow, and collect within the sample chamber (FIG. 1G).

After completion of a drying cycle, the collection vial is removed from the cyclone and the powder is aliquoted into individual containers for storage. The residence time of the aerosolized droplet within the spray dryer was estimated to be approximately 400 ms and is considerably shorter than that of aerosol-assisted EISA (˜3-6 s) performed at a lower Reynolds number or cell-directed assembly (CDA), which requires approximately one minute to achieve complete drying following spin-coating. The outlet temperature measured at the point that particles enter the cyclone remained below 45° C. for the spray drying parameters used in this study. The combined low temperature and short residence time allow for little heat transfer to the droplet, improving conditions for high cell viability.

### Particle Macrostructure and Size Characteristics but not Nanostructure are Dependent on Spray Parameters

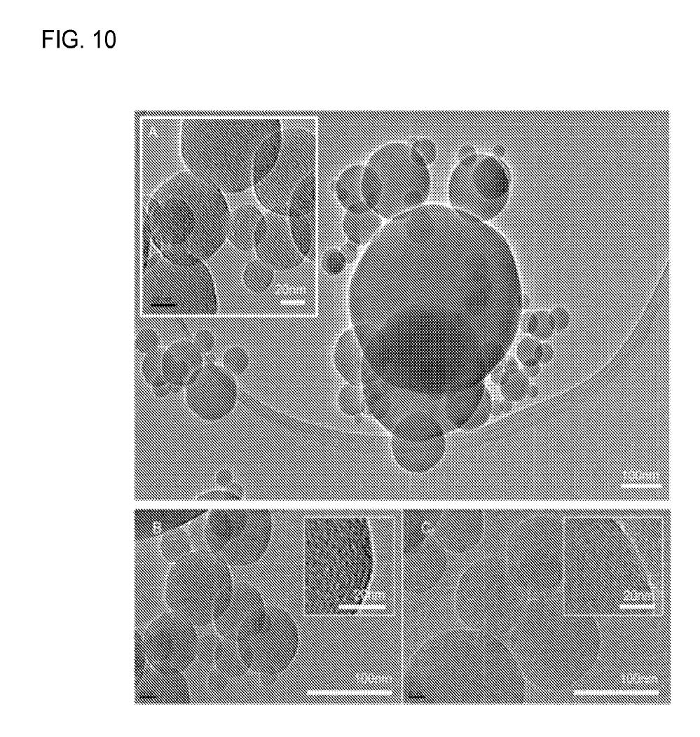

Under all conditions investigated, either latex beads or bacteria were necessary for particle formation and capture; their absence led to no particle accumulation in the collection vial but instead resulted in a thick, dense film on the inside of the cyclone and sample collection chamber. This behavior suggests that, for the rather dilute Precursor Sol used here, the formation of large particles (>1 μm) that can be concentrated and collected in the cyclone requires an effective nucleation site upon which to condense the lipid-silica encapsulant. With or without cells or beads, smaller, spherical nanostructured lipid-silica particles (<1 μm) may form as described for aerosol-assisted EISA but these are drawn by vacuum into the spray drier aspiration filter and are not collected by the spray dryer. To investigate this possibility, aerosol-assisted EISA was performed using the identical Precursor Sol as for spray-drying and an aerosol generator to form ordered, spherical, lipid-silica mesophase particles with sizes ranging from about 20 nm to 1 μm. (See FIG. 10 and associated text for images and experimental details).

Spray drying parameters influenced particle formation and morphology in the cell-containing and bead-containing samples. To establish processing-structure/property relationships, the four independent processing control parameters—nozzle temperature, solution feed rate, sheath gas flow rate, and vacuum aspiration level (Table 1)—were systematically varied and analyzed for effects on macro-morphology, hydrodynamic size, aerodynamic size, and nanostructure (Table 2).

TABLE 2
Macrostructural and nanostructural characteristics of spray-dried biocomposite
particles as a function of spraying parameters Processes A-E.

| | Parameters: | Hollow | | Fine | Geometric | |
|---|---|---|---|---|---|---|
| | Nozzle temp (° C.), | particle | Particle | particle | Standard | |
| | feed rate | fraction | diameter | MMAD | Deviation | Nanostructure |
| Process | (mL/min) | (%)^{A} | (μm) | (μm)^{B} | (μm) | peak (°2θ) |
| A | 60° C., 3.5 mL/min | — | 13.2 ± 1.7 | 6.74 | 1.15 | 2.88 ± 0.04 |
| B | 90° C., 3.5 mL/min | 19.4 | 16.7 ± 4.8 | 3.29 | 1.88 | 2.92 ± 0.01 |
| C | 120° C., 3.5 mL/min | 26.4 | 20.8 ± 2.7 | 3.99 | 2.99 | 2.85 ± 0.06 |
| D | 90° C., 2.5 mL/min | 31.7 | 21.3 ± 2.6 | 2.66 | 1.78 | 2.90 ± 0.02 |
| E | 90° C., 4.5 mL/min | — | 16.8 ± 2.5 | 3.70 | 2.64 | 2.87 ± 0.03 |

^{A}The hollow particle fraction is the observed percentage contribution of hollow particles within a spray-dried sample.
^{B}The Fine Particle Fraction is the fraction of powder from which large, aggregates of particles have been removed.
— Hatch marks indicate that the sample contained no distinguishable hollow particles.
MMAD—Mass Mean Aerodynamic Diameter

Nozzle temperature and solution feed rate were the parameters that most influenced particle characteristics. Nozzle temperatures below 60° C. and feed rates above 4.5 mL/min did not result in the formation of collectible particles. Temperatures exceeding 120° C. were not investigated due to potential heat stresses on the cells. Lower feed rates were not tested due to apparatus limitations. Other process parameters such as sheath gas flow rate and vacuum aspiration level were shown to impact only particle yield and not expanded upon. The process parameters and their qualitative effects on particle formation are summarized in Table 1.

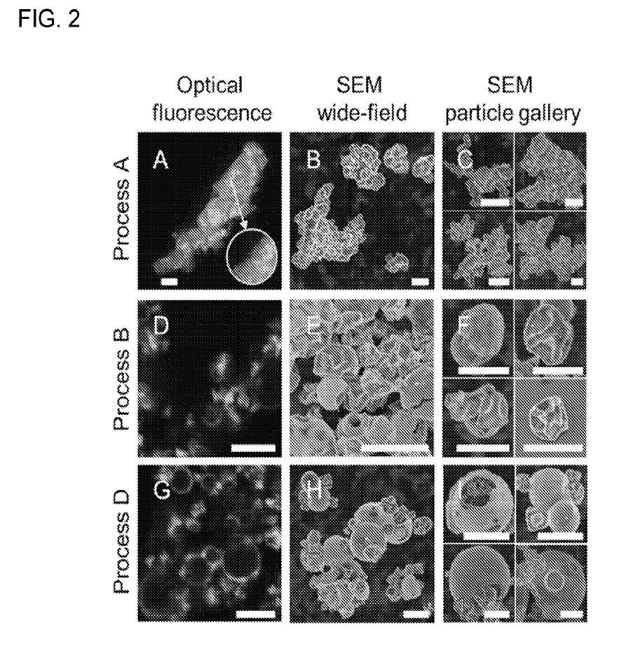

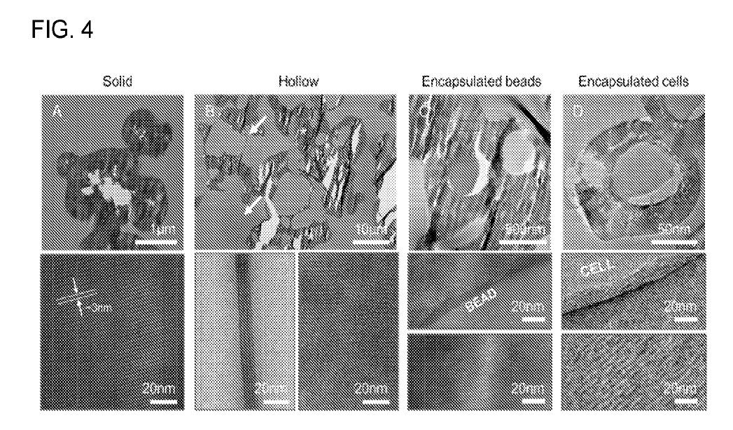

Fluorescence optical microscopy and scanning electron microscopy (SEM) revealed three distinct classes of particles that were dependent on the spraying parameters: large, solid aggregates (FIG. 2A-C); smaller, ‘raisin-like’ solid particles (FIG. 2D-F); and spherical, hollow particles with varying sizes (FIG. 2G-I). Samples that were prepared at low temperature and/or high feed rate (i.e., Process A) typically has large solid particles that are observed to be aggregates of smaller particles. This clumping behavior may be explained by the relatively high moisture content and the large lipid fraction of the biocomposites. Samples that were prepared at high temperature and/or low feed rate (i.e., Process D) had smaller discrete particles with a subpopulation that were hollow and spherical, with a distinct outer shell or crust. However, regardless of macrostructure, both Transmission Electron Microscopy (TEM) (FIG. 4) and low angle X-Ray Diffraction (XRD) (Table 2) revealed a very highly ordered periodic nanostructure that was independent of spray dry conditions. The XRD peak at 20 & 2.9° is consistent with a hexagonal or lamellar lipid-silica mesophase with characteristic d-spacing of 2.5-nm as is formed via EISA of thin films or droplets.

One determinant of particle macromorphology is the Peclet Number (Pe), defined as the (rate of evaporation)/(rate of diffusion), where the evaporation rate and diffusion rate are complex and depend, in turn, on parameters such as temperature, droplet size, concentration, residence time, carrier gas relative pressures in volatile species, etc. (Boissiere et al., 2011, Adv. Mater. 23:599-623; Okuyama et al, 2006, Adv. Powder Technol. 17:587-611). At low Pe (<1) the solutes can diffuse toward the particle center to accommodate the reduced volume resulting from evaporation. This results in smaller, denser particles. At Pe>1 the solute molecules have insufficient time to distribute within the droplet. This results in solute enrichment on the droplet surface. The higher the evaporation rate, the sooner the surface reaches ‘supersaturation’, causing solidification. Further drying creates hollow particles whose size increases and density decreases with increasing Pe. These particles may or may not wrinkle or buckle upon complete drying due to thermal or capillary ‘drying’ stresses. The formation of hollow particles at elevated nozzle temperatures is explained by the more rapid solvent evaporation, which solidifies the exterior of the particle at an earlier stage of drying. After shell formation, the remainder of the precursor solution can continue to self-assemble into an ordered nanostructure on the interior shell wall or collapse into a separate particle enclosed by the hollow particle. The percentage fraction of hollow particles within a sample was determined by counting about 300 or more particles per sample from different SEM images and differentiating between solid (dense and ill-defined shapes) and hollow (spherical) particles. Larger fractions of hollow particles were observed with increasing temperatures and decreasing solution feed rate (data summarized in Table 2).

Both of these conditions increase heat transfer to the droplet surface, thereby increasing Pe and causing solidification of the droplet exterior to occur at an earlier stage of drying when the particle volume is still large. Further solvent removal by diffusion produces hollow particles. Spray drying with lower inlet temperature (e.g., 60° C.) and/or higher feed rate (e.g., 4.5 mL/min) did not result in hollow particles and, in general, yielded larger, solid aggregates. Based on XRD and TEM analysis (see below), after solidification of the particle surface, the remainder of the precursor solution or liquid crystalline mesophase can continue to self-assemble into an ordered nanostructure on the interior shell wall, within a separate particle enclosed by the hollow particle, or within a solid particle (e.g., FIG. 2). Compared to aerosol-assisted evaporation induced self-assembly, the spray-drying process that was used is characterized by a higher Pe (higher inlet temperature and carrier gas feed rate), increasing the likelihood of forming hollow particles. In comparison, for aerosol-assisted EISA, hollow particles have been reported only under limited conditions, for example at high temperature using high volatility solvents (high Pe) or by adding (NH₄)SO₄, which phase separates and thermally decomposes, serving as a ‘bloating’ agent. FIG. 10 shows solid, spherical lipid/silica mesophase particles formed via aerosol-assisted EISA using the identical Precursor Sol as for spray-drying.

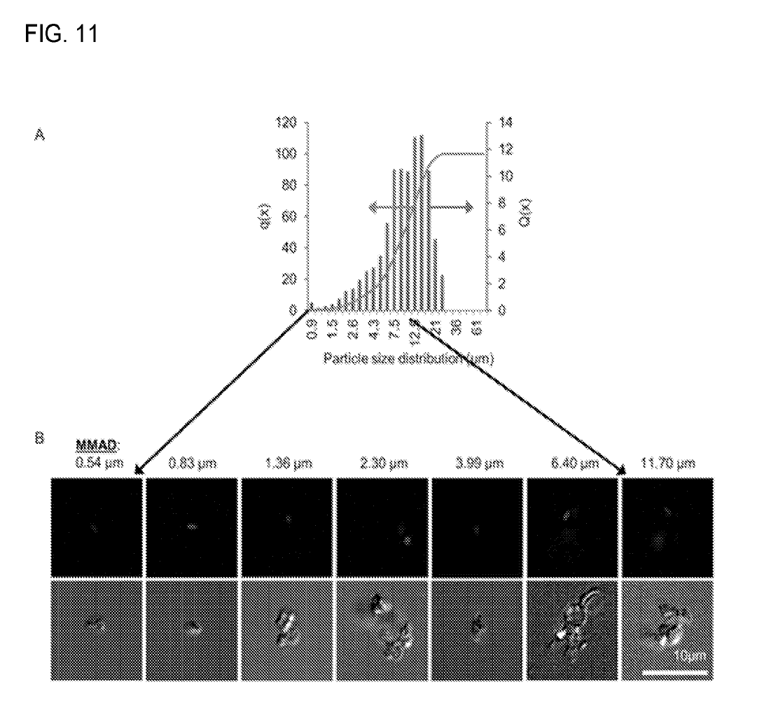

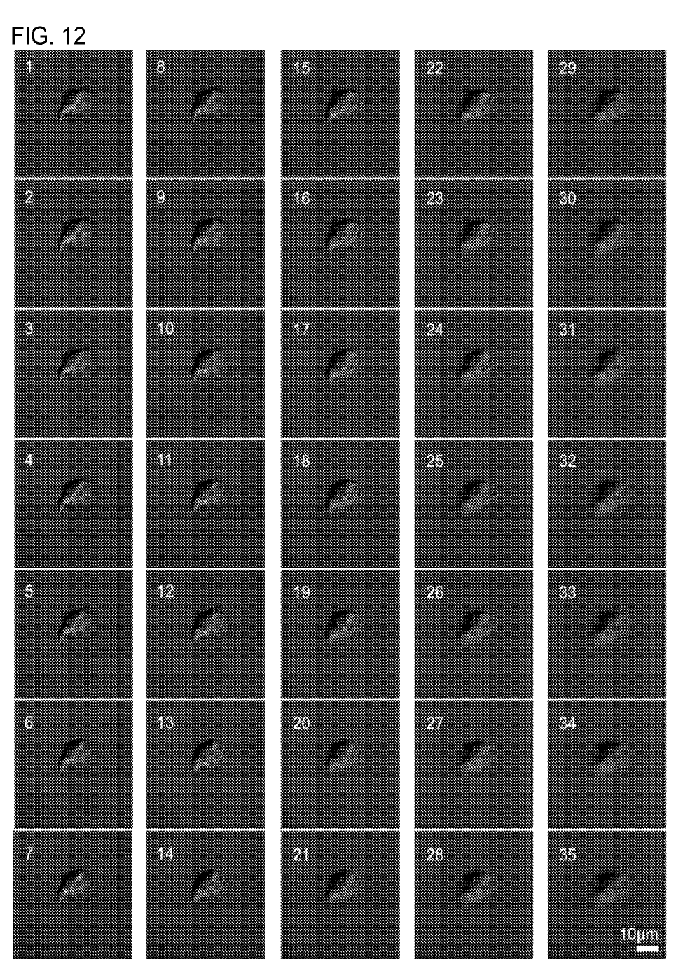

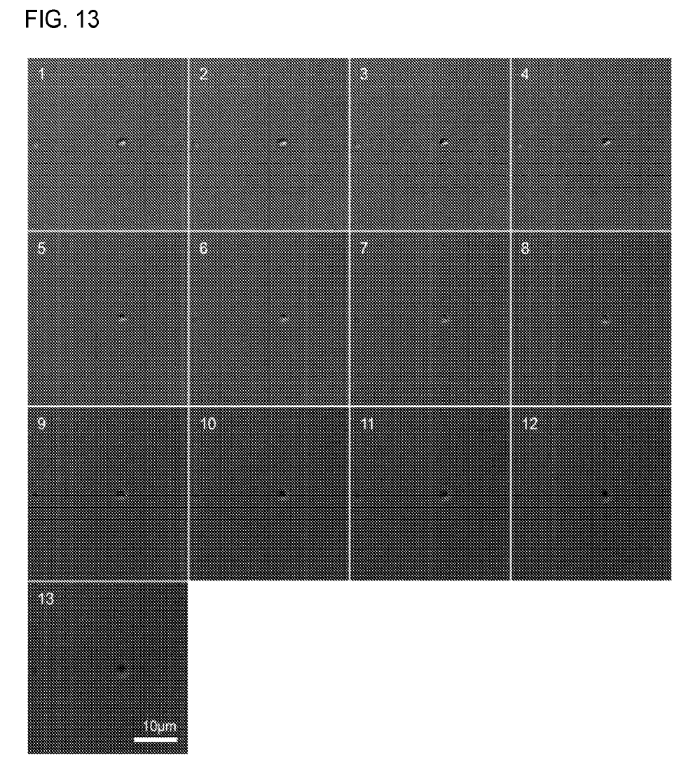

To quantify particle size, powders from each of the Processes were analyzed for particle hydrodynamic size using Laser Diffractometery (FIG. 11A). The Particle Size Distribution and the Geometric Standard Deviation were between 13-21 μm and 1-3 μm, respectively, for all of the Processes tested (Table 2). The aerodynamic properties of dried powders were analyzed by determining the mass mean aerodynamic diameter (MMAD), which is used to simulate dry powder inhalation into and deposition within the lung. This was performed by dry injecting the powders with an insufflator into a steady flow of nitrogen gas flowing through a multi-stage cascade impactor. Particles deposit into different impactor stages according to their aerodynamic diameters and the mass deposited in each stage is used to calculate the effective MMAD. The observed MMAD values represent particles that could be delivered into the deep lung (2.7-6.7 m) for all of the Processes tested (Table 2). These results indicate that biocomposites can be prepared across a wide range of parameters and can result in large particles with high MMADs, small hollow particles with low MMADs, or a mixed distribution of particles. These size properties could allow for aerosol delivery to the deep lung by dry powder inhalation.

Latex Beads and Live Cells are Incorporated into Spray-Dried Biocomposites

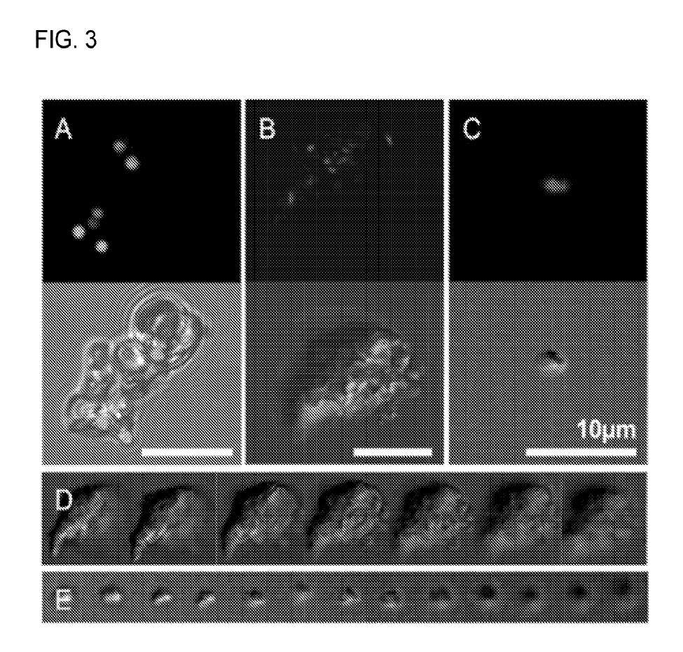

Biocomposites containing encapsulated beads (FIG. 3A) and E. coli (FIGS. 3B-E) were characterized with confocal microscopy to determine the spatial distribution of cells (or beads) within the dried lipid-silica particles. Shown are representative large particles containing multiple beads or cells and a small particle containing one cell. A z-stack image series demonstrated complete encapsulation of cells in both large particles containing many cells with a z-stack depth of 13.0 μm (FIG. 3D) and small particles containing individual cells with a z-stack depth of 6.0 μm (FIG. 3E). To determine the distribution of cells within particles of different aerodynamic sizes, particles were collected by cascade impaction from each well after MMAD-size separation and imaged with confocal microscopy. Cells were found across all of the wells from the largest to the smallest, indicating that particles with MMADs as small as 0.54 μm contain cells (FIG. 11B), which corresponds to particle sizes that could be delivered to the deep lung.

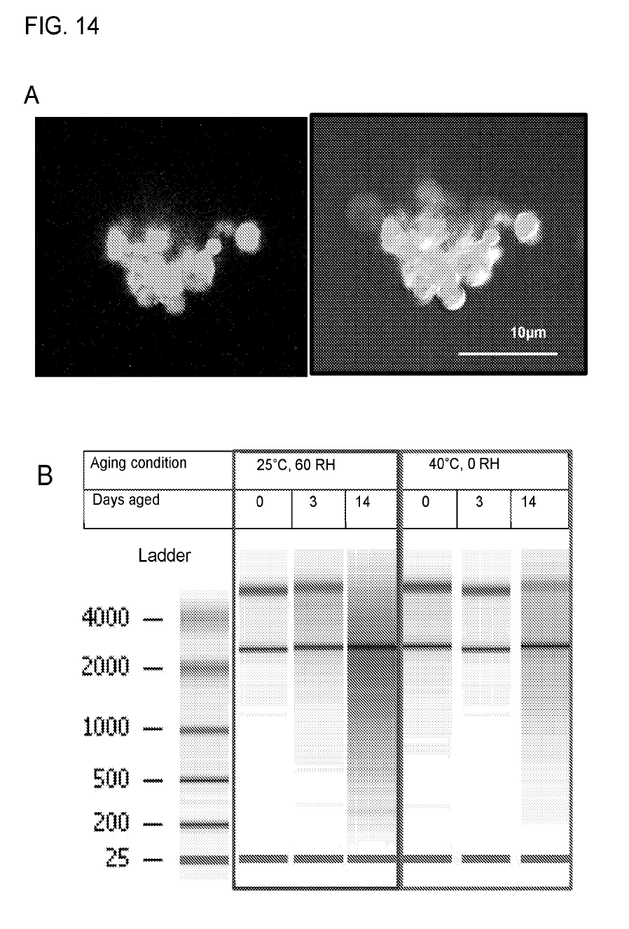

The spray drying process was extended to a eukaryotic cell line by encapsulating yeast within lipid-silica biocomposites. Fluorescence microscopy images of green-stained cells indicate a dense cell loading within dry powder (FIG. 14A) and an RNA isolation assay suggests that yeast maintain intact, purifiable RNA within the encapsulated state (FIG. 14B).

A Ubiquitous Nanostructure Extends Throughout the Particle, is Independent of Spray Parameters, and Interfaces Directly with Encapsulated Cell Walls

Lipid-silica cell encapsulation by cell-directed assembly can involve the formation of a conformal, highly ordered periodic nanostructure that surrounded the cells. This nanostructure can be attributed to EISA, in which solvent evaporation drives the self-assembly of a lipid-silica (polysilicic acid) mesophase, whose fluidity and conformity to the cell surface are enabled by the low heat transfer to the spray-dried composites in the short time scale of the spray dry process. Room temperature aging and progressive condensation of the silica precursor can result in a hardened nanostructure that serves to protect the cell within a hydrophilic matrix that prevents cellular desiccation. It is possible that the shorter processing time and elevated temperature of spray drying compared to, for example, spin-coating may inhibit self-assembly and result in more disordered/non-uniform nanostructures than achieved during spin-coating. In order to examine and characterize the nanostructure of spray-dried powders, low angle XRD and TEM were performed. The XRD samples were prepared simply by loading dry powder onto the XRD sample stage and gently leveling with a microscope slide such that the sample plane was normal to the stage surface. TEM samples were embedded in epoxy and ultra-microtomed into 60-80 nm thick slices following standard procedures.

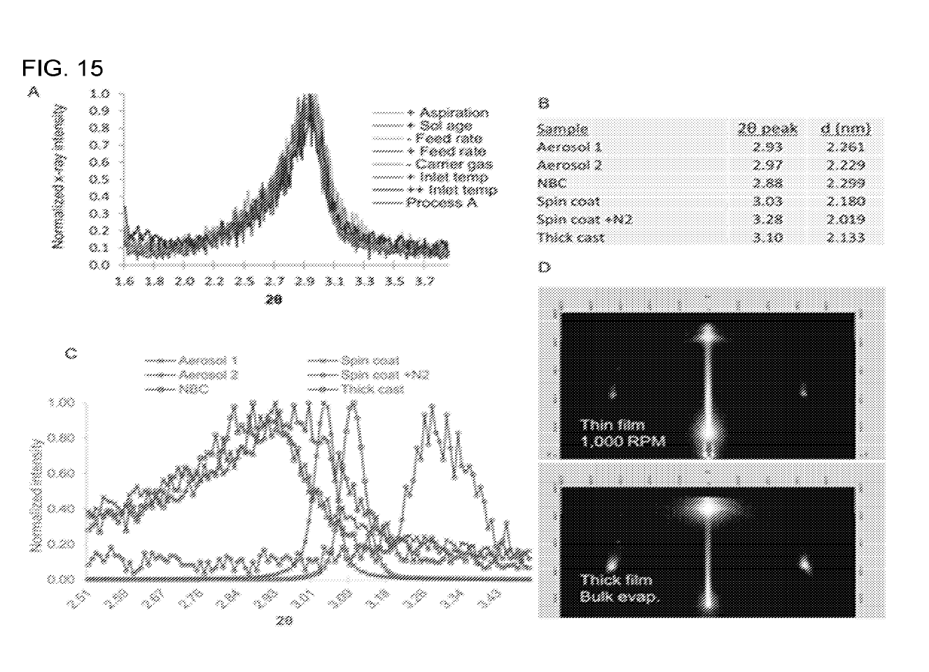

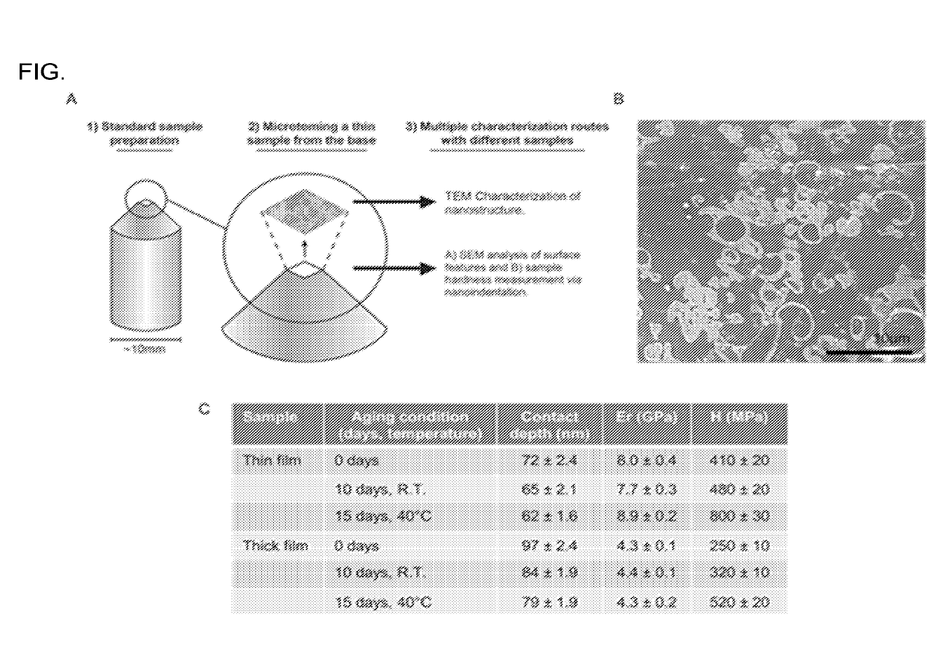

Representative XRD patterns are shown in FIG. 15 and summarized in Table 2 for samples prepared by Processes A-E. All samples produced essentially identical, sharp diffraction peaks centered between 2.85-2.92° 2θ (FIG. 15), corresponding to a consistent nanostructure with lattice d-spacing, d-2.3 nm, according to Bragg's law. This finding indicates that spraying drying yields particles with a well-defined nanostructure that is independent of spraying conditions. For comparison, lipid-silica thin films containing E. coli from the same precursor solutions were prepared by spin-coating according to the published protocol (Baca et al., 2006, Science 313:337-341; Baca et al., 2007, Acc. Chem. Res. 40:836-845). The sprayed dried biocomposites and thin films both have a prominent low angle x-ray diffraction peak (FIG. 15). For films, which are processed at room temperature and have a longer drying time (minutes versus seconds), the XRD peak was narrower and shifted to higher 2θ (3.3° versus 2.9°; FIG. 15B), corresponding to a decrease in d-spacing from 2.3 nm to 2.0 nm). This observation may be due, at least in part, to combined thermodynamic and kinetic effects due to the elevated processing temperature and rapid drying rate associated with spray drying.

Consistent with the XRD results, TEM imaging of thin sections showed a ubiquitous ordered nanostructure that extends throughout the particle independently of spray drying parameters and particle macro-morphology (FIG. 4). Samples containing a homogeneous distribution of solid particles (FIG. 4A, top) or a mixed distribution of solid and hollow particles (FIG. 4B, top) exhibited ordered nanostructures extending throughout the solid regions (FIG. 4B, bottom left) and hollow shells (FIG. 4B, bottom, right). The lattice d-spacing determined by direct measurement of center-to-center dimensions of the ordered nanostructure is approximately 3 nm, consistent with the XRD results. Both stripe patterns and hexagonally close-packed arrays were observed (FIG. 4A-D, lower panels), which may reflect two different orientations of a hexagonal mesophase for the short chain diC₆PC lipid due to its low packing parameter, g, of 1.5-1. One cannot, however, rule out regions of lamellar mesophases. The ‘chattering’ of the microtome cuts evident at lower magnification (FIG. 4, top panels) is attributed to the unexpectedly high modulus and hardness of the lipid silica mesophase. For samples containing either control latex beads or E. coli, the nanostructure is conformal to the surface of the encapsulated object and extends throughout the particle (FIGS. 4C and 4D). Although the disparate hardnesses of the soft cells versus the hardened nanostructure make it hard to preserve intact complete cells in the microtomed samples, the cell/nanostructure interface was located by treating cells prior to spray drying with Osmium tetroxide (OsO₄), which stains the cell membrane with a high Z contrast agent. FIG. 4D highlights the dark rim of the electron dense OsO₄, which is suggestive of an original conformal nanostructure/cellular interface. Due to the sample thickness, the ordered region within the dark rim is attributed to the deeper lying nanostructure that conformed to the 3D cellular interface.

Nanobiocomposites Incorporate Lipids within an Ordered Nanostructure that Maintains Lipid Fluidity for Periods Up to 18 Months Under Dry Storage

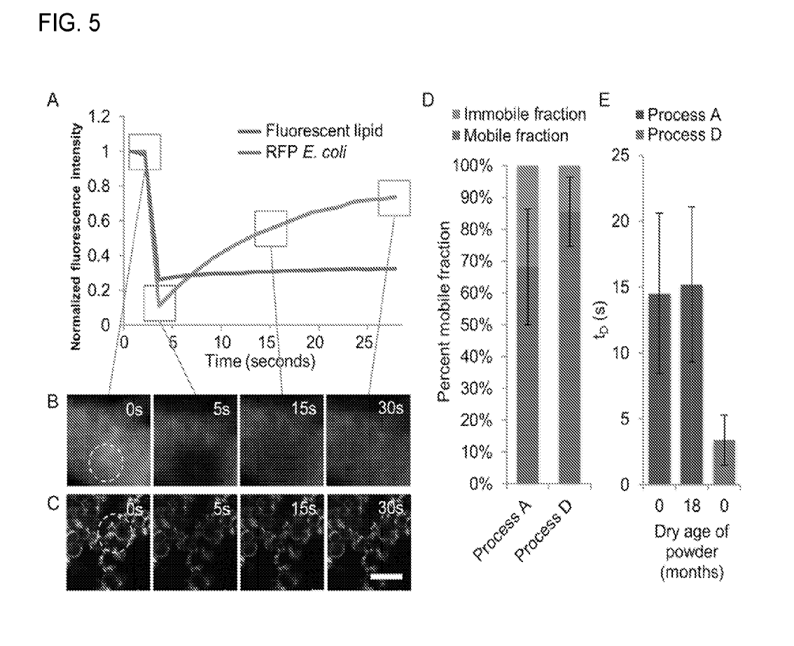

The role of phospholipids during formation and storage of biocomposites is several-fold. First, during cell-directed assembly they direct the formation of a coherent, fluid (liquid crystalline) lipid-silica mesophase that surrounds the cells and is expected to serve as a biocompatible interface that protects them from osmotic, electrostatic, hydrogen-bonding, and drying stresses during solvent drying. Second, the uniform hydrophilic nature of the nanostructured lipid-silica mesophase is expected to retain water by capillary condensation or solvation and thereby prevent cellular desiccation. Third, the nanostructured lipid-silica composite after room temperature aging and further condensation of the silica framework is envisioned to result in a hard mechanical protective shell for the cells that, by virtue of its internal nanostructure, also provides fluid/molecular accessibility to the cell surface. Fluorescence Recovery After Photobleaching (FRAP) was performed to assess the physicochemical state of the lipid fraction during long term storage. FRAP is a process in which fluorescent molecules (here, 1% w/w fluorescently labeled lipids of total lipid fraction) within a small 3D disc-shaped volume are quenched (photobleached) with a high intensity laser pulse and then the region is monitored for fluorescence recovery of intact fluorescent molecules from outside the quenching volume that diffuse into the bleached region (referred to as the mobile fraction). This technique is typically used to characterize membrane component fluidity/diffusivity in cell membranes or lipid vesicles. Analysis of the recovery (shown is a typical recovery curve, FIG. 5A) yields the mobile and immobile fractions of the fluorescent population, which are governed by the equation

R=(F_{∞}−F₀)/(F₁−F₀), (1)

where R is the mobile fraction and F is the fluorescence intensity after full bleaching (F_{∞}), just after bleaching (F₀) and just before bleaching (Fᵢ). FRAP analysis also yields the diffusion time, t_{D}, is defined as the time to recover of ½ the final recovered fluorescent intensity after photobleaching. This is used to calculate the diffusion coefficient, D_{eff} which, for a 2D system, is defined as

$${t}_{D} = \frac{{ω}_{2}^{2} x}{4 {D}_{eff}} , \qquad ( 2 )$$

where ω is defined as the beam radius and y is a correction factor for auto bleaching in the field of view (Reits et al., 2001, Nat. Cell Biol. 3:E145-E147).

Nanobiocomposites containing green fluorescent lipid were prepared according to Process A, which yields larger, more solid particles (FIG. 5B) or Process D, which produces smaller, more hollow particles (FIG. 5C). Powders were suspended in PBS and imaged on a Zeiss LSM 510 confocal microscope. A small region containing a cell was chosen, full laser intensity was applied to a circular bleaching region for approximately two seconds and both red (cell) and green (lipid) fluorescence channels were monitored until the percentage of fluorescence recovery became approximately constant. FIG. 5A is a representative recovery graph for a fresh sample prepared by Process A with red and green fluorescence normalized and corrected for photobleaching. FIG. 5B and FIG. 5C are image progressions of the process before bleaching (0 s), after bleaching (5 s), after half recovery (15 s) and after full recovery (30 s) for particles made from Process A and Process D, respectively (bleaching occurred at approximately two seconds). For Process A, the mobile and immobile fractions were 68±18% and 32±18%, respectively, and for Process D, the mobile and immobile fractions were 86±11% and 14±11%, respectively (FIG. 5D). These values indicate that the majority of the fluorescent species are in the mobile phase and will contribute to fluorescence recovery.

The recovery data was then analyzed for diffusion time in order to determine the diffusion coefficient. The lipid fraction of freshly prepared biocomposites recovered to one-half of the initial fluorescence intensity within 14.5±6.1 seconds and 13.4±1.9 seconds after bleaching, Processes A and Process D, respectively. Assuming a 2D diffusion model, these values correspond to diffusion coefficients of 0.23±0.04 μm²s⁻¹ and 0.8±0.3 μm²s⁻¹ for Processes A and Process D, respectively. Red cellular fluorescence was fully quenched and did not recover due to due to the lack of a source of fresh fluorescent species. This analysis was repeated on samples that were aged for 18 months in a sealed container at room temperature and found that the dry powders maintain fluidity despite the long-term aging (FIG. 5E). The 18-month dry-aged samples were analyzed using the previous 2-D model and exhibited a diffusion coefficient of 0.3±0.2 μm²s⁻¹, statistically similar to the unaged sample. A one-way Anova with post hoc Holm-Sidak testing shows that both Process A samples (0 and 18 months) are significantly different from the Process D sample (P<0.001), but are not significantly different from each other.

Overall these results indicate that the lipid fraction confined within ordered nano-channels—as observed in FIG. 4—retains fluidity and importantly large scale and effective three-dimensional fluidic connectivity as required for fluorescence recovery, which requires lipid diffusion over micrometer length scales. The calculated diffusion coefficients are considerably lower than the diffusion coefficients of GFP in water (87±2 μm²s⁻¹, Potma et al., 2001, Biophys. J. 81:2010-2019) and GFP in the cytoplasm of E. coli (9.0±2.1 μm²s⁻¹, Mullineaux et al. 2006, J. Bacteriol. 188:3442-3448). The previously reported diffusion coefficients are, however, obtained in three-dimensional systems and were calculated using three dimensional models, whereas the data in FIG. 5 was analyzed using a two-dimensional diffusion model that is appropriate for vesicles or supported lipid bilayers. A more appropriate comparison would be for GFP in E. coli periplasm (D_{eff}=2.6±1.2 μm²s⁻¹) and GFP fused to an E. coli plasma membrane protein (D_{eff}=0.13±0.03 μm²s⁻¹) reported by Mullineaux et al. (J. Bacteriol. 188:3442-3448, 2006). The D_{eff} of green-fluorescent lipids observed in the biocomposite matrix described herein (0.23±0.04 μm²s⁻¹) is similar to that reported in the plasma membrane. However if the hexagonal silica nanostructure confines the lipid as from TEM (FIG. 4), the diffusion is in fact quasi-one dimensional and thereby not strictly Brownian.

Nanoindentation Reveals Nanobiocomposites to have Modulus and Hardness Properties Exceeding Mesoporous and Biological Silica Materials

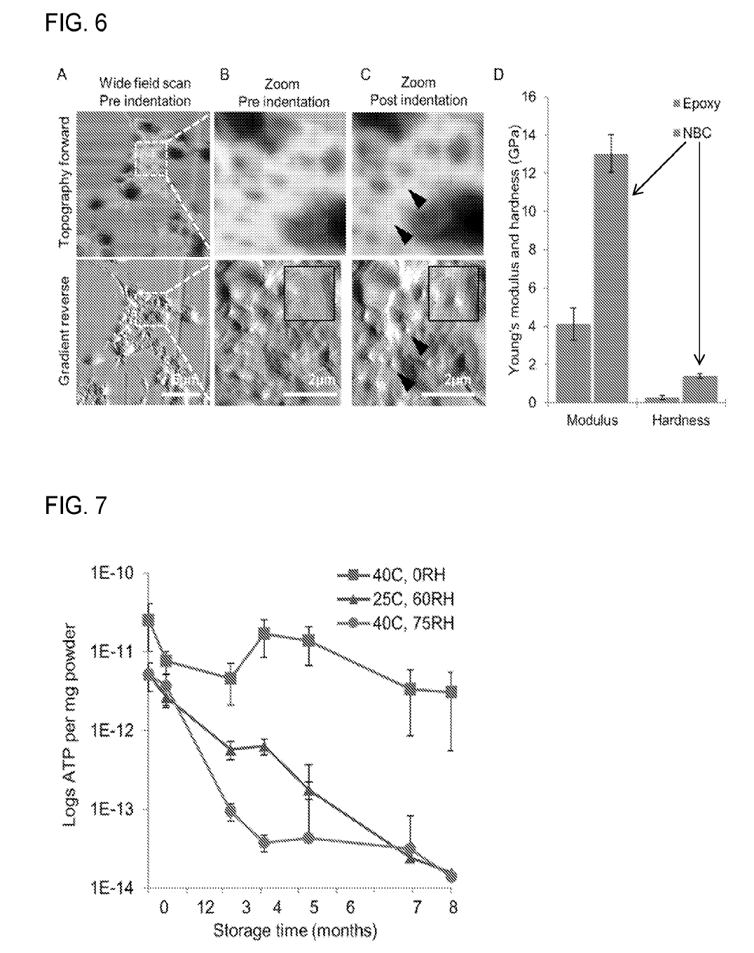

Analyzing the biocomposite nanostructure with TEM involved extensive sample preparation and thin-section preparation with microtoming. Over the course of these experiments, the microtome exhibited blade fatigue, suggesting that the spray-dried particles were unusually hard and tough, especially considering the high lipid content and low temperature processing conditions during sample preparation. Thus, a nanoindentation analysis was performed on biocomposites embedded within an epoxy resin. The nanoindentation analysis was performed on samples from which the TEM thin films were microtomed, which takes the shape of a conical frustum—i.e., a cone with the cap removed (FIG. 16A). The sample was imaged with SEM in backscatter mode (FIG. 16B) with no surface modification to visualize the surface distribution of particles within the epoxy resin. Biocomposites appear white and are clearly distinguishable from the epoxy surroundings (dark gray). Using the same sample, nanoindentation analysis was performed on several particles from different regions on the approximately 1 mm² surface of the substrate (FIG. 6). Shown is a typical particle before (FIGS. 6A and 6B) and after (FIG. 6C) indentation. Indents are marked with black arrows and the diamond-shaped indenting tip is clearly visualized upon magnification (inset). The biocomposites were found to have a Young's Modulus of 13.0±1.0 GPa and a hardness of 1.4±0.1 GPa (n=10). These observed values were significantly greater than the epoxy resin, which exhibited a modulus of 4.1±0.8 GPa and a hardness and 0.3±0.1 GPa. These results indicate that, despite the mild processing conditions, spray-dried biocomposites have Young's modulus and hardness similar to hard biological materials like bone. These values are compared to other related biological and structural materials and summarized in Table 3.

Additionally, Table 3 compares the Young's modulus and hardness of nanobiocomposites to other silicate and biocomposite materials including a hexagonally ordered and oriented 4.5 μm thick-cast film prepared from the identical Precursor Sol as nanobiocomposites using the cell-directed assembly (CDA) methodology. Despite the mild processing conditions, spray-dried nanobiocomposites have Young's modulus and hardness values exceeding those of biological silica, mesoporous silica films, and thick-cast lipid/silica films. Compared to the cell-directed assembly thick-cast lipid/silica films, whose nearly identical hexagonal mesophase is oriented parallel to the substrate and transverse to the indentation direction, the appreciably higher modulus of spray-dried samples may be attributable, at least in part, to the overall 3D orientation of the hexagonal mesophase imposed by confinement of EISA within an evaporating spherical droplet and higher processing temperature, which could promote more extensive silica condensation. With respect to mesophase orientation, cubic mesoporous silica films whose mesopore axes were aligned both parallel and perpendicular to the indentation direction can have higher Young's modulus than hexagonal mesophase films of comparable density whose pore axes were aligned perpendicular to the indentation direction. With respect to potential effects of more extensive condensation on mechanical properties, room temperature aging for 10 days or 40° C. aging for fifteen days can result in significantly increased hardness (320 MPa and 520 MPa, respectively, versus 250 MPa for unaged samples), while having no significant effect on Young's modulus, which remained approximately 4.3 GPa. These values remain considerably lower than for the spray-dried nanobiocomposite materials.

TABLE 3
Young's modulus of biocomposites compared to
similar natural materials and synthetic biomaterials.

| | Young's modulus | |
|---|---|---|
| Material | (GPa) | Hardness (GPa) |
| Nano-Bio-Composites | 13.0 ± 1.0 | 1.4 ± 0.1 |
| CDA thick films, fresh | 4.3 ± 0.1 | 0.250 ± 0.010 |
| CDA thick films, aged 10 days | 4.4 ± 0.1 | 0.320 ± 0.010 |
| at 25° C. | | |
| CDA thick films, aged 15 days | 4.3 ± 0.2 | 0.520 ± 0.020 |
| at 40° C. | | |
| Diatom amorphous silica frustules | 0.347-2.768 | 0.033-0.12 |
| Mesoporous silica | 10-20 | (not reported) |
| (calcined at 500° C.) | | |
| Dehydrated cortical bone | 21.9 ± 3.8 | 0.79 ± 0.19 |
| | 20.02 ± 0.27 | (not reported) |
| Ultra high performance concrete | 48.4 | (not reported) |
| Fused silica glass | 69.64 | 9.22 |
| Nacre aragonite tablets | 92 | 11 |

### NBC Encapsulated Cells as Models of Viable but not Culturable State

The Young's modulus of E. coli is approximately 30 MPa, approximately 500-fold lower than that of its surrounding lipid-silica nanostructure within biocomposites. Thus, the encased bacteria will be physically locked in place within the nanostructure and unable to grow. There is also the potential that the more rigid biocomposite nanostructure could exert significant mechanical stress upon the bacteria during spray drying where capillary (drying) stresses and continued condensation of the silica framework would impose compressive stresses on the cells. This three-dimensional mechanical constraint might induce unique physiological responses in the encased bacteria. A complex relationship exists between mechanical stress and a viable but not culturable (VNBC) microbe: a VBNC can be induced by mechanical stress such as high pressure, while a pre-existing VBNC state can cause resistance to mechanical stress-induced killing. Encapsulated bacterial cells typically show poor viability unless they are first incubated in nutrient-free salt solutions (e.g., PBS) that are known to induce a VBNC state through starvation, consistent with the idea that biocomposite-encased bacteria could be forced into a VBNC state, enabling pre-induced VBNC cells to better survive encapsulation. As used herein, a viable but not culturable (VBNC) refers to cells that exhibit: i) extended retention of markers of cellular metabolism and viability such as ATP levels, and ii) a failure to grow on the routine bacteriological media in which they would normally grow and develop into colonies (Oliver J D, 2005, J. Microbiol. 43:93-100). In some embodiments, the threshold for VBNC character can include a resuscitation rate of 1×10⁴ cells after 10 weeks of encapsulation.

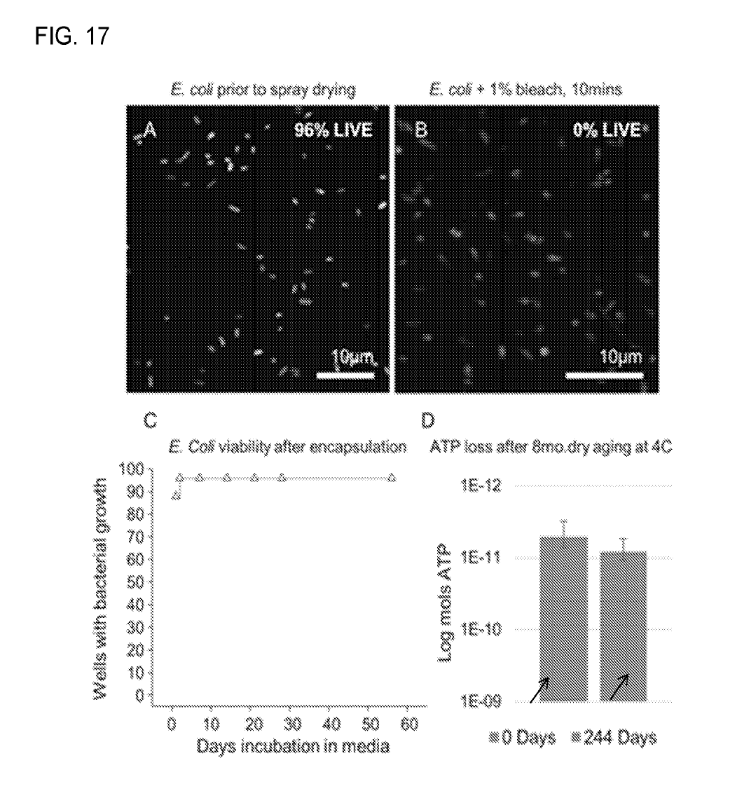

To probe for cellular viability, one can use a luminescence-based adenosine triphosphate (ATP) assay, which is a well-known surrogate indicator for cellular metabolism and viability and has been used in similar live-cell biomaterial research. In this process, ATP is quantified through the ATP-enabled conversion of beetle luciferin to oxyluciferin by firefly luciferase, resulting in a luminescent signal, which is analyzed on a luminometer and is directly proportional to the amount of ATP present. Biocomposites were prepared as described according to Process A and were stored according to published standards on aging of drug and vaccine formulations (U.S. Department of Health and Human Services/Food and Drug Administration. Guidance for Industry: Q1A(R2) Stability Testing of New Drug Substances and Products; 2003) at 4° C. (RH % not specified), 25° C./60% RH, 40° C./75% RH, or 40° C./0% RH. At higher temperatures and humidities, bacterial metabolism will be increased and, in the absence of any nutrients or any ability to benefit from other dead, hydrolyzed cell components (the biocomposite cells are physically isolated from one another and have limited intra-cellular diffusivity), viability and ATP levels of VBNC cells may decrease as is generally observed for cell-based vaccines and other medical products. Biocomposite samples were analyzed periodically for up to eight months and exhibited a strong relationship between ATP retention, time, and storage conditions (FIG. 7). For samples stored at 0% RH, ATP loss was minimal after eight months, whereas it decreased markedly at 25° C./60% RH and 40° C./75% RH. Samples stored at 4° C. were only analyzed at the beginning at end of the experiment due to experimental limitations and showed negligible ATP loss after eight months (FIG. 17).

To determine the effect of sample preparation conditions on ATP, samples were prepared using different spraying parameters described in Table 2 and measured ATP levels as a function of aging at 40° C./75RH or 40° C./0RH. For all processes listed, ATP levels decreased similarly to Process A (Table 4).

TABLE 4

| | | Log loss ATP | Log loss ATP |
|---|---|---|---|
| | Parameters: | after two months at | after two months |
| | Nozzle temp (° C.), | 40° C./75% RH | at 40° C./0% RH |
| Process | Feed rate (mL/min) | (mols) | (mols) |
| A | 60° C., 3.5 mL/min | 2.8 | 0.2 |
| B | 90° C., 3.5 mL/min | 2.2 | 0.1 |
| C | 120° C., 3.5 mL/min | 2.0 | 0.2 |
| D | 90° C., 2.5 mL/min | 2.1 | 0.2 |
| E | 90° C., 4.5 mL/min | 1.9 | 0.3 |

These findings demonstrate the physiological modulation caused by biocomposite encapsulation is independent of spraying conditions. Spray-dried biomaterials are often prepared with excipient materials such as trehalose, sucrose, and/or leucine to reduce cell death resulting from osmotic and drying stresses. Biocomposites were prepared according to Process A and included 15 mM or 100 mM of each excipient separately to the precursors before spraying. Samples were stored for two months at 40° C./75RH and analyzed for ATP. All samples behaved similarly losing between 2.7 and 3.1 logs ATP (Table 5) and so do not improve upon losses observed for control samples (no excipient).

TABLE 5

| | Log loss ATP after two months at 40° C./75% RH |
|---|---|
| Excipient added | (mols) |
| 5 mM trehalose | 3.1 |
| 15 mM trehalose | 2.6 |
| 5 mM sucrose | 3.1 |
| 15 mM sucrose | 2.8 |
| 15 mM leucine | 2.7 |
| Growth media | 3.8 |

This suggests that the progressive replacement of water with the conformal, hydrophilic lipid-silica nanostructure (FIG. 4) during spray drying maintains a biocompatible nano/bio interface. Furthermore, the addition of liquid growth media to spray-dried biocomposites resulted in a noticeably greater decline in ATP with a loss of 3.8 logs ATP after two months (Table 5), which may be attributed to how increased metabolism of encapsulated cells reduces their ability to cope with stresses of cellular confinement. Induction of a VBNC state by physical and chemical cellular confinement within a rigid biocompatible nanostructure is consistent with our observations.

In addition to sustained ATP levels, the VBNC state is characterized by a significant reduction in the ability of the VBNC cells to enter back into normal cell growth and division, a process termed resuscitation. Quantifying the numbers of VBNC cells capable of resuscitation can be challenging, however. Although plating on solid media allows for colony counting from isolated progenitor VBNC cells, many bacteria show much less ability to grow from such states on solid media compared to liquid media, exemplified by Mycobacterium tuberculosis that is capable of extended VBNC or latency. Biocomposites may provide a model of VBNC bacteria and their resuscitation that would enable temporal separation of the VBNC state and subsequent resuscitation states. In this model, the VBNC state occurs in the solid phase encapsulating matrix and resuscitation then occurs in liquid phase culture after dissolution of the silica. To test this, the frequency of cellular resuscitation in media after increasing periods in the biocomposite-induced VBNC state was measured. E. coli expressing RFP were spray-dried and aged at room temperature and humidity for zero to 36 weeks. The total number of cells spray-dried was divided by the total collected amount of powder to yield an approximate cell-loading quantity. Aliquots of biocomposite with an estimated specific cell count were then serially diluted in PBS containing 20% Fetal Bovine Serum (FBS) and dispensed in 96-well plates so that each well would contain the same average number of cells. Dilutions ranged from 10⁰ to 10⁵ cells/well/plate. Plates were capped, sealed with adhesive tape to prevent evaporation, and incubated at 37° C. with moderate rotary agitation for eight weeks. Plates were analyzed periodically for bacterial fluorescence from RFP that indicates regrowth (resuscitation) using a fluorescence plate-reader and, for visual confirmation, a digital camera and UV-transilluminator.

FIG. 17C shows a time course of resuscitation for samples aged for two weeks and dispensed at a concentration of 100 cells per well. Resuscitation occurred rapidly and nearly completely. This level of resuscitation correlates well with the approximately 96% viability of E. coli immediately prior to encapsulation as determined with live/dead staining (FIGS. 17A and 17B).

Regrowth occurred in wells in a rapid but stochastic manner and was consistent with a control experiment in which overnight growth of a single cell led to a positive signal. Thus, resuscitation of a single cell from the VBNC state to growth is sufficient to result in overnight growth to turbidity and a positive RFP signal. The frequency of this resuscitation event can then be determined from its occurrence as a function of total initial colony forming units (CFU) added. Low frequency resuscitation will only be apparent at high CFUs per well.

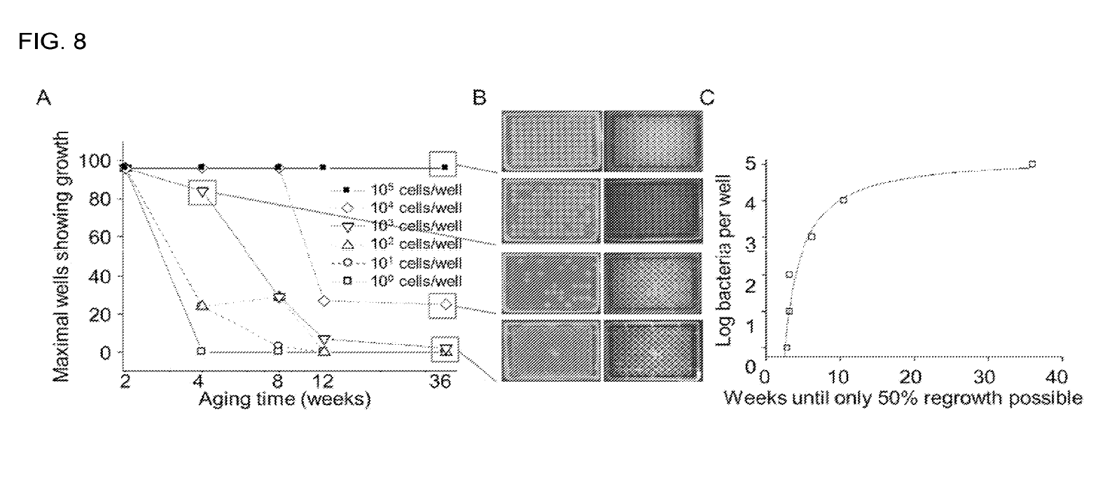

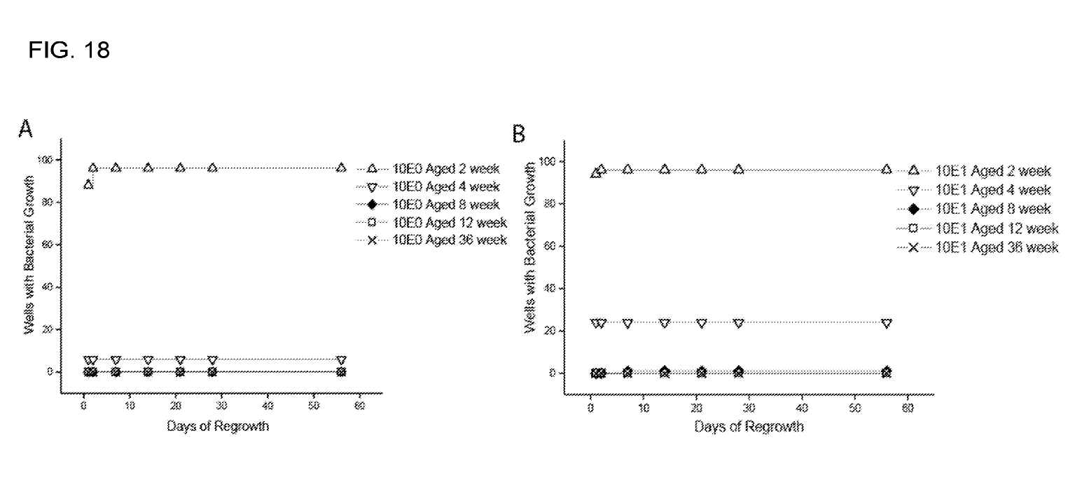

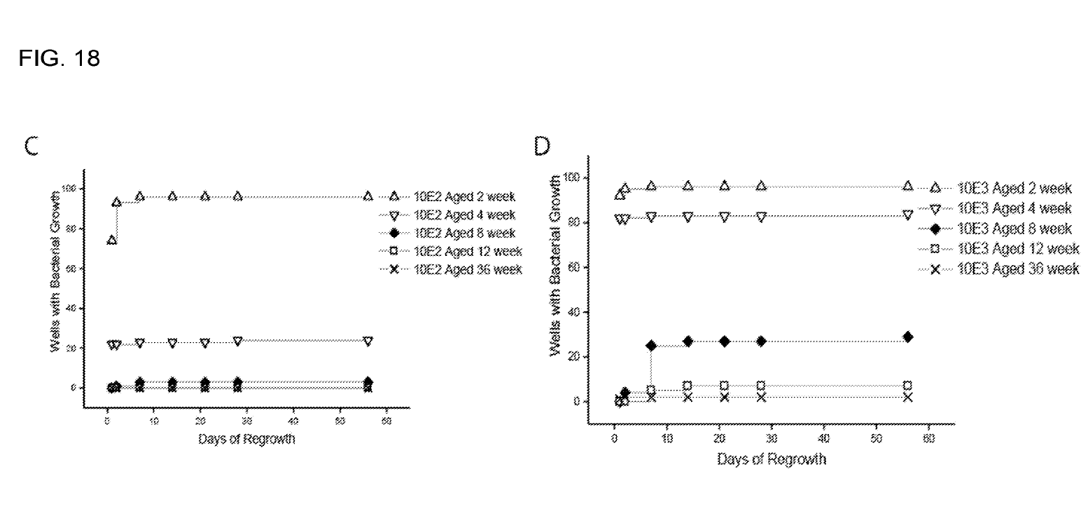

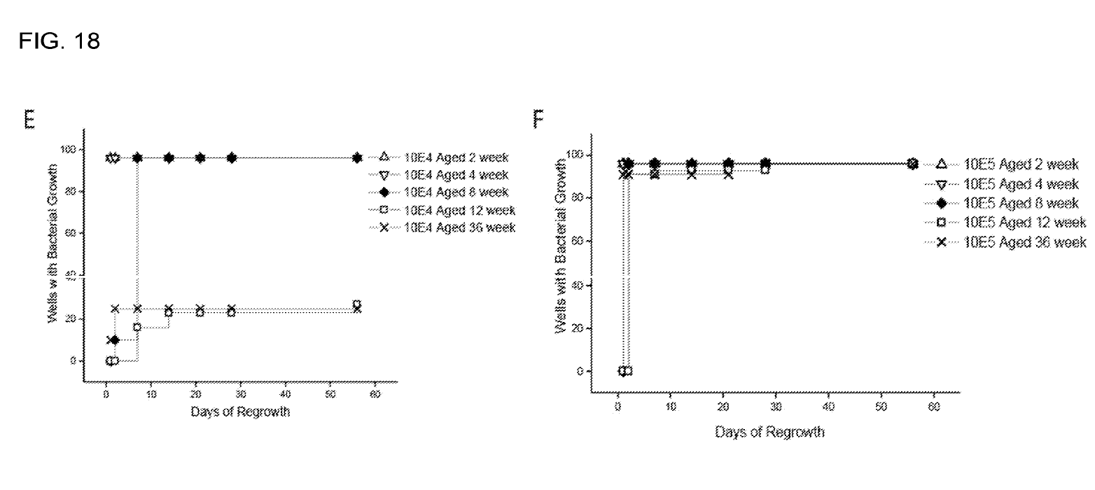

The number of wells exhibiting regrowth as a function of CFU and liquid incubation time is shown for biocomposite-aging times of 0-36 weeks in FIG. 8. The frequency of resuscitation decreased with the time in the VBNC state. FIG. 18A-F demonstrates that, generally, most positive resuscitation in each well occurred in the first few days of liquid media incubation. However, FIG. 18D-F show that at greater periods of biocomposite-induced VBNC, resuscitation could take up to four weeks of liquid culture, but the frequency of such late resuscitation events was low, as it was only observed for >10³ CFU/well.

The maximal number of wells showing growth (after 56 days liquid media culture) is shown as a function of the length of biocomposite-induced VBNC (aging) in FIG. 8A. Representative digital images and plate reader images are shown in FIG. 8B. As the aging time increased, the number of wells with growth decreased in a manner that is dependent upon the initial CFU. Thus when low initial CFU were present, the ability for resuscitation in any well was lost early. At greater CFU, the probability of a resuscitation event increased and was possible even after extended periods of biocomposite-induced VBNC. The fraction of at least 50% of the wells undergoing resuscitation as a function of aging time and initial CFU is shown in FIG. 8C, demonstrating that the frequency of resuscitation after periods of aging exceeding about 10 weeks is rare (less than 1 in 10⁴ cells). Combined, the high preservation of cellular ATP, but very low frequency of resuscitation of biocomposite-encased bacteria is consistent with a large population in the VBNC state. This approach enables one to determine resuscitation probabilities from VBNC using a high throughput platform, even when such events are rare.

In addition to enabling basic studies of bacterial resuscitation, this platform could also be used to screen drugs that are capable of killing pathogens that exist in a VBNC state. Exemplary diseases caused by such pathogens include, but are not limited to, tuberculosis and melioidosis. Thus, this platform may be used to screen drugs capable of killing pathogens such as, for example, Mycobacterium tuberculosis, Burkholderia pseudomallei, Staphylococcus aureus, or pathogens that form biofilms. The platform similarly may be sued to screen drugs against cancer stem cells—e.g., non-replicating or slowly replicating cancer cells that are typically impervious to conventional chemotherapeutics.

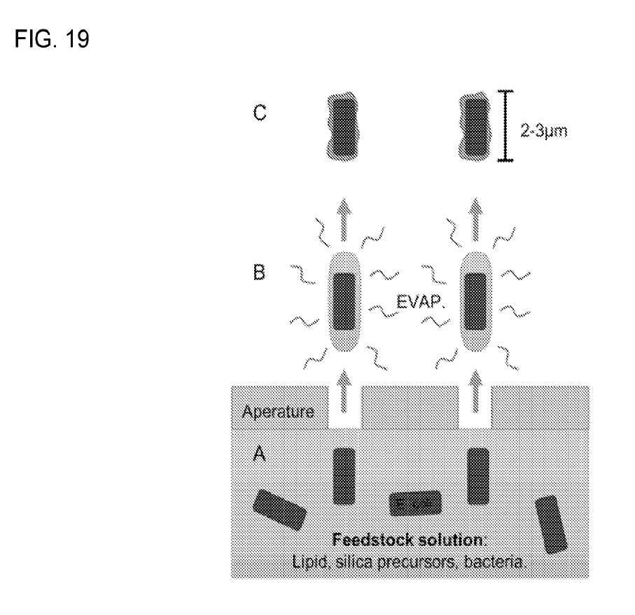

In some embodiments, the nanobiocomposites particles may be aerosolized. This method processes 0.3 mL/min of feedstock and produces a homogeneous population of particles with geometric size distribution of 2-3 μm and a Mass Mean Aerodynamic Diameter (MMAD) of 3.4 μm (FIG. 19). These materials—and the techniques used to produce the materials—may be relevant to the fields of pulmonary drug and vaccine delivery and/or treating cellular dormancy.

The nanobiocomposite can exhibit a minimum MMAD of at least 0.5 μm such as, for example, at least 1.0 μm, at least 1.2 μm, at least 1.4 μm, at least 1.6 μm, at least 1.8 μm, at least 2.0 μm, at least 2.2 μm, at least 2.4 μm, at least 2.6 μm, at least 2.8 μm, at least 3.0 μm, at least 3.2 μm, at least 3.3 μm, at least 3.4 μm, at least 3.5 μm, at least 3.6 μm, at least 3.7 μm, at least 3.8 μm, at least 3.9 μm, at least 4.0 μm, at least 4.2 μm, at least 4.4 μm, at least 4.6 μm, at least 4.8 μm, at least 5.0 μm, or at least 6.0 μm. The nanobiocomposite can exhibit a maximum MMAD of no more than 10 μm such as, for example, no more than 8 μm, no more than 7.5 μm, no more than 6.8 μm, no more than 6.0 μm, no more than 5.0 μm, or no more than 4.0 μm. In some embodiments, the nanobiocomposite can exhibit an MMAD expressed as a range having endpoints defined by any minimum MMAD listed above and any maximum MMAD listed above that is greater than the minimum MMAD. In some embodiments, the nanobiocomposite can exhibit an MMAD of from 2.6 μm to 6.8 μm such as, for example, from 2.6 μm to 4.0 μm.

Spray dried nanocomposites exhibit long-lasting biological properties including ATP stability and culturability post encapsulation. A vibration-aerosolization method was adapted to exploit these biological preservative effects. Vibrating mesh aerosolizers provide an alternative manufacturing process that one can use to prepare nanobiocomposite that involve lipid/silica encapsulation of living cells. Here, powders containing E. coli were manufactured and characterized. This new manufacturing method is much less expensive and significantly easier than the previous spray drying methods, allowing for a more accessible material preparation technique, while maintaining advantages of the spray drying method—e.g., high volumes of powders with robust biological preservation properties.

In some embodiments, the nanobiocomposite can include one or more lipids or other biocompatible surfactant such as, for example, those used in the pharmaceutical and cosmetics industry. Moreover, the solubility of a silica coating can be modulated by doping the silica sol with one or more soluble titania precursors to result in a silica/titania shell. For example, a silica shell will naturally dissolve at pH greater than 2. This solubility can be controlled by doping the silica with materials such as titania. The silica sol can be doped with any suitable amount of titania precursor such as, for example, a ratio of at least 80 mol % silicon to 20% mol % titanium. In one exemplary embodiment, the silica sol may be doped using a ratio of 95 mol % silicon:5 mol % titanium. A silica shell also is naturally hydroscopic/hydrophilic and will begin to dissolve in environments with ambient humidity greater than about 30% relative humidity. This can be controlled by adding a hydrophobic component (e.g., a silane) either to the silica sol or after powder formation. A hydrophobic moiety can be added up to the point of making the resulting materials float when placed in a variety of liquids or further increased to the point that the silica shell becomes superhydrophobic, which would result in a layer of air between the silica shell and any aqueous liquid phase in which materials could be immersed.

The nanobiocomposite can further include additional compounds besides a surfactant and the shell material. For instance, it may be useful to add nutrients or sugars in order to control metabolism of cells (see Carnes et al., 2010, Nature Chem Bio 6:41-45).

In summary, this disclosure describes a new technique that involves using a spray drying approach for the scalable production of lipid-silica biomaterials with fully encapsulated, live cells. The spraying conditions can provide for brief cell-solvent contact times, low operating temperatures, and/or rapid droplet drying rates, allowing for cell viability in otherwise harsh material preparation conditions. The nanocomposites can have a highly ordered nanostructure independent of spray-dry conditions, incorporating a fluid lipid interphase that is retained after 1.5 years of storage at room temperature, yet they are rigid and hard.

The nanocomposite can be sufficiently rigid to exhibit a Young's modulus of from 0.1 MPa to 100 GPa. Thus, the nanocomposite can exhibit a minimum Young's modulus of at least 0.1 MPa such as, for example, at least 1 MPa, at least 10 MPa, at least 25 MPa, at least 50 MPa, at least 100 MPa, at least 250 MPa, at least 500 MPa, at least 1 GPa, at least 5 GPa, at least 10 GPa, at least 20 GPa, at least 30 GPa, at least 40 GPa, or at least 50 GPa. Also, the nanocomposite can exhibit a maximum Young's modulus of no more than 100 GPa such as, for example, no more than 50 GPa, no more than 25 GPa, no more than 10 GPa, no more than 5 GPa, no more than 1 GPa, no more than 500 MPa, no more than 250 MPa, no more than 100 MPa, no more than 50 MPa, no more than 25 MPa, no more than 10 MPa, no more than 5 MPa, or no more than 1 MPa. In some embodiments, the nanocomposite can exhibit a Young's modulus expressed as a range having endpoints defined by any minimum Young's modulus listed above and any maximum Young's modulus listed above that is greater than the minimum Young's modulus. Thus, for example, a nanocomposite can exhibit a Young's modulus of from 1 MPa to 50 GPa. In some embodiments, a nanocomposite can exhibit a Young's modulus of from 10 GPa to 20 GPa such as, for example, 10 GPa, 11 GPa, 12 GPa, 13 GPa, 14 GPa, 15 GPa, 16 GPa, 17 GPa, 18 GPa, 19 GPa, or 20 GPa. In one particular example, a nanocomposite can exhibit a Young's modulus of 13 GPa.

The nanocomposite can be sufficiently hard to exhibit a hardness of 0.01 MPa to 100 GPa. Thus, a nanocomposite can exhibit a minimum hardness of at least 0.01 MPa such as, for example, at least 0.1 MPa, at least 0.5 MPa, at least 1 MPa, at least 5 MPa, at least 10 MPa, at least 50 MPa, at least 100 MPa, at least 250 MPa, at least 500 MPa, at least 1 GPa, at least 2 GPa, at least 5 GPa, or at least 10 GPa. Also, the nanocomposite can exhibit a maximum hardness of no more than 100 GPa such as, for example, no more than 10 GPa, no more than 5 GPa, no more than 2 GPa, no more than 1 GPa, no more than 500 MPa, no more than 250 MPa, no more than 100 MPa, no more than 50 MPa, no more than 25 MPa, no more than 10 MPa, no more than 5 MPa, no more than 2 MPa, no more than 1 MPa, or no more than 0.5 MPa. In some embodiments, the nanocomposite can exhibit a hardness expressed as a range having endpoints defined by any minimum hardness listed above and any maximum hardness listed above that is greater than the minimum hardness. Thus, for example, a nanocomposite can exhibit a hardness of from 0.1 MPa to 10 GPa such as, for example, from 1 GPa to 2 GPa. Thus, in some embodiments, a nanocomposite can exhibit a hardness of 1.0 GPa, 1.1 GPa, 1.2 GPa, 1.3 GPa, 1.4 GPa, 1.5 GPa, 1.6 GPa, 1.7 GPa, 1.8 GPa, 1.9 GPa, or 2.0 GPa. In one particular example, a nanocomposite can exhibit a hardness of 1.4 GPa.

These unique properties appear to induce the VBNC state, although resuscitation is possible after even extended periods. The materials provide a model for determining VBNC resuscitation frequencies across a wide range of variation. While viability and nanostructure appear independent of spray drying parameters, particle macro-morphology, density, and aerodynamic diameters were variable through systematic control of the processing parameters. Such low-MMAD particles containing VBNC-bacteria could prove useful for the development of live, attenuated vaccines with enhanced expression of VBNC-related antigens and provide potential as a vaccine against, for example, latent tuberculosis.

Accordingly, the nanocomposite may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

A pharmaceutical composition may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the nanocomposite, its use in the pharmaceutical compositions is contemplated. Supplementary active ingredients also can be incorporated into the compositions. As used herein, “pharmaceutically acceptable” refers to a material that is not biologically or otherwise undesirable, i.e., the material may be administered to an individual along with the nanocomposite without causing any undesirable biological effects or interacting in a deleterious manner with any of the other components of the pharmaceutical composition in which it is contained.

Thus, a nanocomposite may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the nanocomposite into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A pharmaceutical composition that includes a nanocomposite material as described herein may therefore be administered to a subject having or at risk of having a condition treatable with the nanocomposite material. As used herein, “at risk” refers to a subject that may or may not actually possess the described risk. Thus, for example, a subject “at risk” of infection by a microbe is a subject present in an area where individuals have been identified as infected by the microbe and/or is likely to be exposed to the microbe even if the subject has not yet manifested any detectable indication of infection by the microbe and regardless of whether the subject may harbor a subclinical amount of the microbe.

In this aspect, the nanocomposite material may be used to treat a subject having or at risk of having a condition. As used herein, “treat” or variations thereof refer to reducing, limiting progression, ameliorating, or resolving, to any extent, the symptoms or signs related to a condition. “Symptom” refers to any subjective evidence of disease or of a patient's condition, while “sign” or “clinical sign” refers to an objective physical finding relating to a particular condition capable of being found by one other than the patient.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

In another aspect, the nanobiocomposites can be used to coat, for example, urease-producing bacteria and/or fungi so that they can be incorporated into self-repairing concrete formulations. The long-lasting urease-producing microorganisms can provide a self-repairing function to a structure manufactured from concrete that includes the nanobiocomposite, resulting in less cost for maintenance, repair, and/or replacement of concrete structures. Moreover, the VBNC microbe in the nanobiocomposite can survive for a much longer period than without the nanobiocomposite, resulting in a more durable self-repair character. Similarly, microbes that metabolize and/or produce carbonates can be encapsulated to produce, for example, self-healing concrete or drywall.

In another aspect, the nanobiocomposites can be used in a preparation of methane consuming microbes. The preparation may be applied so that the microbes can consume methane in order to, for example, bioremediate a methane plume. Such a preparation also may be used to consume methane released from tundra or other natural sources.

In another aspect, the nanobiocomposites can be a way of encapsulating probiotic organisms—e.g., either a single cell type or multiple cell type cocktail—for oral delivery of the probiotic organisms. The probiotic organism may be formulated, as described in more detail above, into, for example, a tablet or capsule. The silica shell, which is naturally stable at stomach pH (pH 2), can provide protection for the encapsulated materials in the stomach but begin to dissolve and release the probiotic organisms when passed into the intestinal tract, where the pH increases to 5.5-7.4. Additional protection from stomach contents could be provided by, for example, adding titania to silica shell and/or adding a hydrophobic moiety. This protection would additionally be beneficial for any oral application, including potentially enabling orally-administerable vaccines.

In yet another aspect, the nanobiocomposites can be a way of encapsulating organisms commonly used as pesticides such as, for example, Bacillus thuringiensis. In addition to providing protection and controllable dissolution, the particle size can also be varied to produce materials with controlled aerosol dissemination properties optimized for distribution methods—e.g., hand-held sprayer, fogger, or aerial crop-duster.

Finally, while described herein in the context of exemplary embodiments in which the nanobiocomposite is a component of a spray dried powder, the nanobiocompoistes may be used in other forms. For example, the nanobiocomposite may be spray-coated—e.g., using a plant sprayer—to coat an irregular surface, clothes, equipment, a vehicles, etc. This would enable, for example, the use of cells as sensors built into a surface with the potential to actively respond to analytes, creating a smart sense-and-respond materials, a self-cleaning material, and/or a self-protecting materials. In some embodiments, the nanobiocomposite may be applied using a technique that allows one to scalably (e.g., continuously produce a uniform layer. Spin and dip coating (see, e.g., Carnes et al., 2006, Science 313:337-341) can produce uniform layers but is not scalable in many instances.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Materials.

Leucine, trehalose, Carbenicillin disodium salt, Lennox Broth (LB), agar, acetonitrile, ethanol (absolute), Tetraethyl orthosilicate, and hydrochloric acid were purchased from Sigma Aldrich (St. Louis, Mo.). Short-chain 1,2-dihexanoyl-sn-glycero-3-phosphocholine (diC₆PC) and 1-hexanoyl-2-{6-[(7-nitro-2-1,3-benzoxadiazol-4-yl)amino]hexanoyl}-sn-glycero-3-phosphocholine (06:0-06:0 NBD PC) were obtained from Avanti Polar Lipids (Alabaster, Ala.). Fluorescent biomarker SYTO 9 green fluorescent permeable nucleic acid stain was obtained from Invitrogen (Life Technologies. Carlsbad, Calif.). 1 μm Fluoromax fluorescent latex beads were obtained from Thermo Scientific. Nitrogen (N₂) was purchased from a local supplier (Argyle gas, Albuquerque, N. Mex.).

### Cell Culture.

Bacteria (K12 Escherichia coli, strain BL21) was purchased from Sigma Aldrich (St. Louis, Mo.) and transformed with pDsRed-Express 2 (Clontech, Mountain View, Calif.), which constitutively expresses DSRed-Express2, a highly stable Red Fluorescence Protein variant, and confers resistance to ampicillin or carbenicillin for cell selection. Cells were grown in LB (20 g/μL) containing 100 μg/mL carbenicillin for 12 hours at 37° C. with shaking until an OD₆₀₀ of 1.2-1.5 was reached, corresponding to approximately 1.5×10⁷ cells/mg biocomposite. Prior to subsequent sample preparation, cells were washed three time by pelleting at 4,000 RPM for five minutes and resuspended in PBS.

### Silica Precursors and Precursor Sol.

Prehydrolyzed tetraethyl orthosilicate stock solutions (A2**) were prepared following previously used methods (Baca et al., 2006, Science 313:337-341) by refluxing 61 mL of TEOS, 61 mL of ethanol, 4.9 mL of DI water and 0.2 mL of 0.07 N HCl (molar ratio 1:4:1:5×10⁻⁵) for 90 minutes at 60° C. Stock solutions were stored at −20° C. The silica Precursor Sol was prepared by adding 0.8 3 mL A2** stock to a solution containing 1.3 mL DI water, 0.66 mL ethanol and 0.53 mL 0.07 N HCl. This solution was allowed to age at room temperature with sonication for 30-60 minutes until complete condensation had occurred (see FIG. 9). Immediately prior to sample preparation, 100 mg lipid was added to the fully condensed solution and mixed until fully dissolved (˜20 seconds). This is the final, active lipid-silica precursor solution that referred to as Precursor Sol.

### Preparation and Storage of Spray-Dried Biocomposites and Thin Film Analogues.

Samples were spray-dried with a Mini Spray Drier B-290 (Büchi, Flawil, Switzerland) using a 0.7 mm nozzle. Initial processing conditions were defined as Process A and consisted of 60° C. inlet temperature, 90% aspiration rate, 3.5 mL/min peristaltic pump feed rate, and 60 μL/hr nitrogen carrier gas rate. 3.3 mL of Precursor Sol and 3.3 mL of cells in liquid suspension were loaded into separate scintillation vials. Two peristaltic pumps with a combined feed rate of 3.5 mL/min were used to deliver the solutions to the nozzle with mixing through a Y connector immediately prior to inspiration into the nozzle.

Spray-dried particles were collected in scintillation vials that were connected to the standard cyclone with a custom-built adapter, replacing the standard collection chamber. Sample yields were approximately 150 mg per batch, corresponding to approximately 1.5×10⁷ cells/mg powder. After spray drying, samples were stored at 4° C., 25±2° C./60±5% RH, 40±2° C./75±5% RH, or 40±2° C./0±5% RH according to published aging standards.³⁸ For comparison to previous studies in cell-directed assembly, thin film samples were prepared according to published techniques (Baca et al., 2006, Science 313:337-341; Baca et al., 2007, Acc. Chem. Res. 40:836-845).

For comparison to previous studies in cell-directed assembly, thin films were prepared by spin coating, thick films were prepared by bulk solution evaporation (casting), and aerosolized samples were prepared by aerosol-assisted EISA according to published techniques (Baca et al., 2006, Science 313:337-341; Baca et al., 2007, Acc. Chem. Res. 40:836-845).

### Characterization of Particle Morphology and Size.

To determine the physical structure of biocomposites, samples were prepared under different conditions with varying inlet temperatures and feed rates as described in Table 1. The samples were analyzed and observed with SEM, confocal microscopy, and TEM. The percentage fraction of hollow particles within a sample was determined by counting >300 particles per sample in SEM images and differentiating between solid and hollow particles.

The hydrodynamic diameter, D₅₀, of dried powders was measured using a Sympatec HELOS laser diffractometer (Clausthal-Zellerfeld, Germany). 1-2 mg of powder was suspended in 1 mL of water, sonicated for 10 seconds to break up particle agglomerates, vortexed for 30 seconds to distribute individual particles, and the vial was left to rest for 60 seconds to allow additional aggregates to settle out of suspension. 100 μL of suspension was pipetted into the LD cuvette containing 6 mL of acetonitrile, mixed thoroughly, and data was collected. The Fine Particle Fraction Mass Mean Aerodynamic Diameter was determined using a Next Generation pharmaceutical cascade Impactor (NGI, Copley Scientific Ltd., Nottingham UK). Powders were dry-injected into the cascade impactor using a DP-4 dry powder insufflator (Penn-Century, Inc., Wyndmoor, Pa.) to disperse the individual particles. A pump maintained a steady flow through the NGI to simulate inspiration (30 L/min). Particle clumping was observed during the pressurized aspiration process, which may have caused particles to be forced together and is attributed, at least in part, to the large lipid fraction. This behavior was accounted for by subtracting out the weight of the sample that deposited within the largest well of the cascade impactor (well #1), allowing us to sort out large aggregates of particles that, in vivo, would deposit within the upper respiratory tract of the lung. The effective MMAD value represents the effective size of particles that would be delivered into the deep lung and fell within 2.6-6.8 μm for all of the Processes tested (Table 2).

### Optical Microscopy.

For optical imaging, dried powders were suspended in water, vortexed for 10 seconds and pipetted onto standard microscope slides. Samples were imaged on a Zeiss LSM 510 confocal microscope mounted on a Zeiss Axiovert 100 inverted microscope. Latex beads are phosphorescent (excitation and emission peaks are 468 nm and 508 nm), yeast were stained with Syto-9 green fluorescent dye according to manufacturer's specifications, and E. coli samples constitutively express an RFP variant (excitation and emission peaks are 554 nm and 591 nm) and were not further fluorescently treated.

A gallery of z-stack images for particles of varying sizes were prepared in order to visualize the distribution of cells within particles. This was achieved by setting the upper and lower boundaries of a particle and taking an image with a given optical slice diameter (here 0.4 m and 0.5 μm) and collecting an image every diameter distance. The resulting collection of images maps the entire z-dimension within the sample, allowing us to create 3D reconstructions of the sample.

### Electron Microscopy.

Scanning Electron Microscopy was performed using a Hitachi S-5200 Nano SEM operating between 1-5 kV. Spray-dried biocomposites were distributed onto a SEM sample boat coated in carbon tape and seated into the tape with a short pulse of N₂ gas. No further sample preparation was performed for imaging.

Transmission Electron Microscopy was performed using a Hitachi H7500 TEM equipped with an AMT XR60 bottom mount camera or on a JEOL 2010F field emission HRTEM/STEM with HAADF detector. biocomposites containing beads or E. coli were suspended overnight in PBS at 4° C., fixed in 2.5% glutaraldehyde in PBS overnight at 4° C., washed three times in PBS, fixed in 1% osmium tetroxide (only for samples containing E. coli cells), washed three times in water, dehydrated in a graded ethanol series, and switched to an anhydrous acetone for the final dehydration. The preparation was then infiltrated with resin by incubating particles in 1:1 Spurr's resin:acetone, 3:1 Spurr's resin:acetone and, finally, 100% Spurr's resin. Samples were placed in embedding molds, polymerized by incubation at 60° C. for at least 16 hours, and the blocks were trimmed for microtoming. Microtomed sections with thicknesses between 60 nm and 80 nm were used for imaging.

### Measurement of Lipid Fluidity.

Fluorescence Recovery After Photobleaching (FRAP) was used to measure lipid fluidity using the confocal set-up as described above. Sample preparation was the same as previously described and included 1% w/w NBD-labeled C-6 PC lipid (added to Precursor Sol along with C-6 lipid). Powders were spray-dried, collected, and re-suspended in PBS immediately prior to imaging. FRAP was performed by photobleaching a region on a particle and measuring the following fluorescence recovery. Auto bleaching was measured in an adjacent, unbleached region and used as a correction factor for in the FRAP recovery data. A large particle was selected for analysis to allow for accurate measurement of the recovery rates by ensuring a large quantity of excess fluorophores.

### Nanoindentation Characterization of Biocomposite Modulus and Hardness.

Nanoindentation was performed on a nanoindentor (TRIBOINDENTER, Hysitron, Inc., Eden Prairie, Minn.) with a cube-corner tip. Nanoindentation was performed using the pyramidal shaped epoxy-resin substrate that was used for the TEM experiments. During biocomposite indentation the contact radius was kept small so that the plastic zone beneath the tip (approximately three times contact radius) was contained within the biocomposite with minimal influence from the epoxy-resin substrate. A fused quartz standard was used to determine the indenter tip area function as a function of contact depth. Control indents were performed in the epoxy regions surrounding the encapsulated particles. Young's modulus and hardness for both biocomposite and epoxy indents were determined via the Oliver-Pharr method (Oliver W C and Pharr G M, 2004, J. Mater. Res. 19:3-20).

### ATP Assay.

Cell viability was measured using an ATP-based luminescence assay (Bactiter Glo, Promega Corp., Madison, Wis.). After storage under the above-mentioned conditions, a measured amount (5-10 mg) of dry powder was resuspended in water to a 1 mg/25 μL dilution. The solution was thoroughly mixed and 25 μL was added to wells in a 96-well plate. The Bactiter reagent was prepared according to product literature and 50 μL reagent was added to each sample well and the plate was analyzed on a luminometer (Tecan Group Ltd., Mannedorf, Switzerland). The data was normalized to ATP standards (containing 10⁻¹² to 10⁻¹⁶ mols ATP). The data is representative of four experiments. As a control, encapsulated beads were analyzed and found to be below our limit of detection.

### Culturability Assay.

Nanobiocomposite samples were freshly prepared, sealed in an air-tight vial, and dry aged at room temperature for 2 weeks, 4 weeks, 8 weeks, 12 weeks, or 36 weeks prior to culturing experiment. At the start of the regrowth experiment, 96-well plates were prepared such that the same weight of powder containing an approximate cell/mg loading as described above was loaded into each of 96 wells in a 96-well plate such that each well had the same approximate number of cells. First, a set of serial dilutions of cells in media were prepared. The media used consisted of 20% FBS containing carbenicillin, which was prepared immediately before the experiment. For the cell dilutions, 1.2 mg of biocomposite was added to 1.2 mL of media (1 mg/mL), 0.12 mL of the remaining sample was added to 1.08 mL of media (0.12 mg/1.2 mL=0.1 mg/mL), and so forth. This dilution set, therefore, consists of biocomposites in media with approximately 10⁷ cells/mL, 10⁶ cells/mL, etc.

Second, the cell/media solution was added to the 96 wells on a plate. 1 mL of the first dilution containing 10⁷ cells/mL was added to 9 mL of media as described above in a small vial. This solution was stirred continuously with a stirplate/stirbar throughout the following preparation to ensure a well-mixed product. 100 μL containing 10⁵ cells is pipetted into each well of the first 96-well plate and the remaining 400 μL was discarded.

Third, the plate was capped and sealed around the perimeter with adhesive tape to prevent evaporation. This method was shown to contain liquid media for significantly longer than the duration of the experiment (data not shown). The plate was then set aside and the remaining cell dilutions were prepared in the same way. The final set of samples were seven 96-well plates containing 10⁵ cells/well/plate, 10⁴ cells/well/plate, 10³ cells/well/plate, 10² cells/well/plate, 10¹ cells/well/plate, 100 cells/well/plate, and a control plate in which 1 mL of PBS was substituted for the 1 mL of cell dilution added to 9 mL of media.

The seven plates were sealed in a container as a further prevention against evaporation and incubated at 37° C./60 RPM for eight weeks. Each day for the first week and weekly thereafter, each plate was imaged using a fluorescence plate reader with excitation and emission filters set to DsRed fluorescence (554 nm and 591 nm respectively). For visual clarity, plates also were imaged using a digital camera with excitation from a UV-transilluminator. The above procedure was then repeated periodically such that regrowth data points occurred at 2 weeks, 4 weeks, 8 weeks, and 32 weeks of dry sample aging.

### Example 2

### Production of Aerosolized Particles.

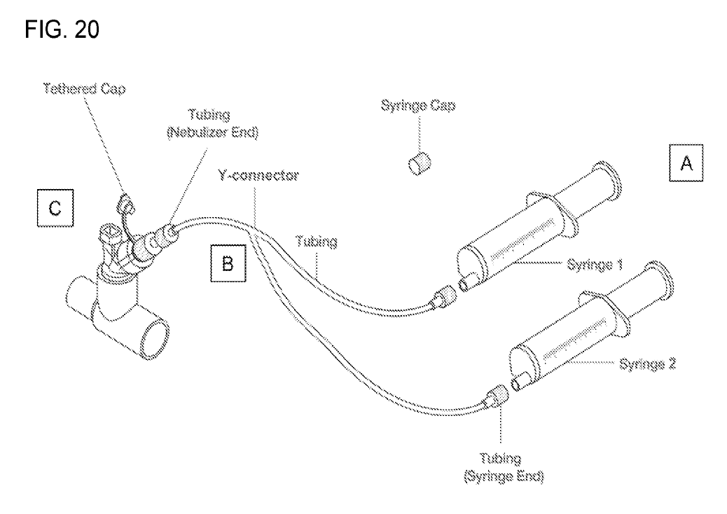

Nano-bio-composites (NBCs) are prepared using vibration induced aerosolization (VIA) using a commercially available unit (Aerogen, Ltd., Galway, Ireland). A silica Precursor Sol is prepared by adding 0.83 mL A2** stock to a solution containing 1.33 mL DI water, 0.66 mL ethanol and 0.53 mL 0.07N HCl for a total Sol volume of approximately 3.3 mL. This solution is allowed to age at room temperature with sonication for 30-60 minutes until complete condensation had occurred. Immediately prior to sample preparation, 100 mg lipid is added to the fully condensed solution and mixed until fully dissolved, approximately 20 seconds. This is the final, active lipid-silica precursor solution that we refer to as the precursor Sol and it is loaded into Syringe 1 (FIG. 20). E. coli are grown to an optical density of 1.2-1.5 OD₆₀₀, washed three times, resuspended in PBS, and 3.3 mL are loaded into Syringe 2. The two syringes are manipulated with an automatic multi-syringe dispenser set at 0.15 mL/min for a total of 0.3 mL/min feed rate. We run the apparatus for ˜20 minutes using 3.3 mL of each solution. The total collection of VAP powder is ˜100 mg. This technique allows us to produce scalable quantities of powders with highly-defined MMADs suitable for drug or vaccine delivery to the lung. The short cell-solvent contact time as achieved using the continuous delivery syringe model and low processing temperatures allows for high post processing cell viability.

Post processing, dried particles are collected and stored in scintillation vials under the following conditions to simulate drug and pharmaceutical compound aging: 4° C., 25±2° C./60±5% RH, 40±2° C./75±5% RH, and 40±2° C./0±5% RH.³⁸ These samples are analyzed as a function of storage conditions for materials properties, as described in Example 1.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A biocomposite material comprising:
a cell; and
a lipid-silica matrix at least partially encapsulating the cell.

2. The biocomposite material of claim 1 wherein the cell is viable but not culturable (VBNC).

3. The biocomposite material of claim 1 wherein the cell comprises a pathogen.

4. The biocomposite material of claim 1 wherein the lipid-silica matrix comprises a dried sol.

5. The biocomposite material of claim 4 wherein the lipid-silica matrix comprises an ordered nanostructure.

6. The biocomposite material of claim 5 where the ordered nanostructure is characterized by an X-ray diffraction peak at 2θ of 1-4 degrees.

7. The biocomposite material of claim 6 where the ordered nanostructure is characterized by an X-ray diffraction peak at 2θ of 2-3 degrees.

8. The biocomposite material of claim 1 comprising a mass mean aerodynamic diameter (MMAD) of from 2.6 μm to 6.8 μm.

9. A vaccine comprising the biocomposite of claim 1.

10. The vaccine of claim 9 wherein the biocomposite is aerosolized.

11. A method of making a biocomposite, the method comprising:
providing a cell;
aerosolizing a mixture of lipid and silica precursor;
combining the cell and the aerosolized mixture, thereby allowing the aerosolized mixture to at least partially encapsulate the cell; and
allowing at least a portion of the aerosolized mixture to evaporate.

12. The method of claim 11 wherein allowing at least a portion of the aerosolized mixture to evaporate induces evaporation-induced self-assembly of the lipid and silica precursors in the aerosolized mixture.

13. The method of claim 11 further comprising drying the partially-encapsulated cells to form a powder.

14. A method comprising:
providing a biocomposite material of claim 1;
contacting the biocomposite material with a test compound; and
evaluating at least one effect of the test compound on the cell.

15. The method of claim 14 wherein the cell comprises a pathogen.

16. A method comprising:
administering the vaccine of claim 9 to a subject having or at risk of having a condition treatable by the biocomposite material.
